(19) United States
(12) Patent Application Publication
(10) Pub. No.: US 2005/0256042 A1
(43) Pub. Date: Nov. 17, 2005

(54) Methods of preventing and treating alimentary mucositis

(76) Inventors: Michael E. Jeffers, Branford, CT (US); Zachary Yim, Glen Gardner, NJ (US); Pascal Valax, Chernex (CH); Pradyumna Kumar Namdev, Emeryville, CA (US); Rajeev A. Chillakuru, Chester, CT (US); William J. LaRochelle, Madison, CT (US); Henri S. Lichenstein, Guilford, CT (US); Enrique Alvarez, Clinton, CT (US); William Hahne, Madison, CT (US)

Correspondence Address: Jenell Lawson;Intellectual Property, CuraGen Corporation, 555 Long Wharf Drive, New Haven, CT, 06551, US

(21) Appl. No.: 10/980,459

(22) Filed: Nov. 3, 2004

Related U.S. Application Data
(63) Continuation-in-part of application No. 10/435,087, filed on May 9, 2003.
(63) Continuation-in-part of application No. 10/842,179, filed on May 10, 2004.
(60) Provisional application No. 60/541,728, filed on Feb. 4, 2004.
(60) Provisional application No. 60/545,278, filed on Feb. 18, 2004.

(51) Int. Cl. A61K 38/17; C07K 14/47; C12N 1/21; C12P 21/06; C12N 15/74

(52) U.S. Cl. 514/12; 435/69.1; 435/320.1; 435/252.33; 530/350

(57) ABSTRACT

The present invention relates to compositions and methods for preventing and treating alimentary mucositis. More particularly, the present invention provides methods for preventing and/or treating alimetary mucositis by using compositions comprising FGF-20, a fragment, a derivative, a variant, a homolog, or an analog thereof.

This application is a continuation-in-part of the U.S. patent application Ser. No. 10/435,087, filed May 9, 2003, and Ser. No. 10/842,179, filed May 10, 2004. This application also claims the benefit of U.S. Provisional Application Nos. 60/541,728, filed Feb. 4, 2004, 60/545,278, filed Feb. 18, 2004, and Ser. No. ______, Attorney Docket No. Cura-57 OM4, filed Oct. 28, 2004, entitled “COMPOSITIONS AND METHODS OF USE FOR A FIBROBLAST GROWTH FACTOR.” The content of each is incorporated herein by reference in its entirety.

### 1. FIELD OF THE INVENTION

The present invention relates to compositions and methods for preventing and treating alimentary mucositis. More particularly, the present invention relates to compositions comprising FGF-20, a fragment, a derivative, a variant, a homolog, or an analog thereof, and their uses in preventing and treating alimentary mucositis.

### 2. BACKGROUND OF THE INVENTION

### 2.1 Alimentary Mucositis

Alimentary mucositis refers to a form of mucosal barrier injury to the alimentary tract. Alimentary mucositis may occur at a part or multiple parts of the alimentary tract, from mouth to anus, via, e.g., esophagus, stomach, small intestine, colon, and rectum. Non-limiting examples of alimentary mucositis are oral mucositis, esophagitis, stomatitis, enteritis, and proctitis. See, e.g., Blijlevens et al., Bone Marrow Transplant 25:1269-1278 (2000); and Keefe et al., Seminars in Oncology 20:38-47 (2004).

Alimentary mucositis are generally caused by one or more insults, most commonly by a chemical(s) or radiation, or a combination thereof. Radiation may be a result of, e.g., radiation therapy, accidental radiation exposure, and radiation exposure from a terrorist attack. See e.g., Moulder, Int. J. Radiat. Biol. 80:3-10 (2004). Chemical insults are commonly from chemotherapy.

Oral mucositis commonly occurs as a painful, dose-limiting toxicity of chemotherapy and radiation therapy, especially in cancer patients. The disorder is characterized by, e.g., breakdown of the oral mucosa that results in the formation of ulcerative lesions. In myelosuppressed patients, the ulcerations that accompany mucositis are frequent portals of entry for indigenous oral bacteria, which often lead to sepsis or bacteremia. Oral mucositis occurs to some degree in more than one-third of patients receiving anti-neoplastic drug therapy. The frequency and severity are significantly greater among patients who are treated with induction therapy for leukemia or with many of the conditioning regimens for bone marrow transplant. Among these individuals, moderate to severe oral mucositis is not unusual in more than three-quarters of patients. Moderate to severe mucositis occurs in virtually all patients who receive radiation therapy for tumors of the head and neck and typically begins with cumulative exposures of 15Gy and then worsens as total doses of 60Gy or more are reached (See, e.g., Peterson, Curr Opin Oncol 11(4):261-266 (1999); Plevova, Oral Oncol. 35(5):453-470 (1999); Knox et al., Drugs Aging 17(4):257-267 (2000); and Sonis et al., J. Clin Oncol 19(8):2201-2205 (2001)).

Current standard care for oral mucositis is predominantly palliative, including application of topical analgesics such as lidocaine and/or systemic administration of narcotics and antibiotics (See, e.g., Peterson, Curr Opin Oncol 11(4):261-266 (1999); Plevova, Oral Oncol. 35(5):453-470 (1999); Knox et al., Drugs Aging 17(4):257-267 (2000); Sonis et al., J. Clin Oncol 19(8):2201-2205 (2001)). Several agents have been evaluated for safety and efficacy in preventing or treating oral mucositis (See e.g., Peterson, Curr Opin Oncol 11(4):261-266 (1999); Plevova, Oral Oncol. 35(5):453-470 (1999); Knox et al., Drugs Aging 17(4):257-267 (2000); Rosenthal et al., Antibiot. Chemother. 50:115-132 (2000); Crawford et al., Cytokines Cell Mol. Ther. 5(4):187-193 (1999); Bez et al., Oral Surg. Oral Med. Oral Pathol. Oral Radiol. Endod. 88(3):311-315 (1999); and Danilenko, Toxicol Pathol 27(1):64-71 (1999)). These include mucosal protective agents, antibiotics, transforming growth factor (TGF), interleukin-11(IL-11), granulocyte-macrophage colony stimulating factor (GM-CSF), and keratinocyte growth factor (KGF).

The main clinical manifestations of esophagitis (mucositis of esophagus) are dysphagia (difficulty in swallowing), odynophagia (painful swallowing), substernal (in radiation-induced esophagitis) and/or retrosternal (in chemotherapy-induced esophagitis) chest pain. See e.g., Keefe et al., Seminars in Oncology 20:38-47 (2004). Esophagitis is commonly managed by topical use of local anesthetics, spasmolytis, analgesic drugs, and treatment of acid reflux. Ranitidine or omeprazole has been recommended for the prophylactic reduction of epigastric pain and heartburn following certain chemotherapies. In some instances, the severity of the esophagitis requires a temporary interruption of the treatment or modification of the treatment plan. See e.g., Keefe et al., Seminars in Oncology 20:38-47 (2004).

Stomatitis (mucositis of stomach) may be radiation-induced and is characterized by, e.g., dyspepsia and symptomatic gastritis, which are usually temporary. In some instances, gastric ulcer may occur. Not many reports have been published on chemotherapy-induced stomatatitis. Currently, histamine-2 receptor antagonists or proton pump inhibitors are commonly used to manage the symptoms associated with stomatitis. See e.g., Keefe et al., Seminars in Oncology 20:38-47 (2004); Sartori, J. Clin. Oncol. 18:463-467 (2000).

Enteritis (mucositis of intestines, especially the small intestine) is common in patients who receive abdominal or pelvic radiation therapy, cytotoxic agents, or a combination thereof. The main symptoms are nausea, abdominal pain, bloating, and diarrhea. Radiation-induced diarrhea typically occurs during the first two weeks after beginning of radiation therapy. The mechanism of radiation-induced diarrhea involves acute mechanical damage to the epithelial crypt cells of the gastrointestinal tract. Such damage results in cell death (necrosis), inflammation, and ulceration of the intestinal mucosa, which is then exposed to irritating bile salts and becomes susceptible to opportunistic infections. See e.g., Gwede, Seminars in Oncology Nursing 19:6-10 (2003). Chemotherapeutic agents that commonly associated with diarrhea include, but are not limited to, fluoropyrimidines (e.g., 5-fluorouracil), topisomerase I inhibitors (e.g., irinotecan, topotecan), and other agents (e.g., cisplatin, oxaliplatin, cytarabine). See e.g., Viele, Seminars in Oncology Nursing 19:2-5 (2003). Chronic bowel toxicity may also occur after radiation therapy, usually six months to three years after the therapy. Patients often have intermittent constipation and diarrhea, which may cause malnutrition and disturbance of electrolytes. In severe cases, acute intestinal obstruction, fistulas, or bowel perforation may occur. See e.g., Keefe et al., Seminars in Oncology 20:38-47 (2004).

Recent studies suggest that p53 and p21, two transcription factors, may play an important role in chemotherapy-induced enteritis. See, Pritchard et al., Clin. Cancer Res. 6:4389-4395 (2000); Pritchard et al., Cancer Res. 58:5453-5465 (1998); Bilim et al., J. Exp. Clin. Cancer Res. 19:483-488 (2000); and Potten et al., stem Cells 15:82-93 (1997). The Bcl-2 family of proteins may also contribute to the sensitivity of the small intestine to most chemotherapy agents. See e.g., Keefe et al., Seminars in Oncology 20:38-47 (2004).

Mucositis of the colon is characterized by, e.g., diarrhea and crampy abdominal pain. Chronic injury of the colon has symptoms of intermittent diarrhea and constipation caused by fibrotic strictures and pseudo-obstruction. Irinotecan (CPT-11) has been reported to cause significant diarrhea and hypothesized severe colon damage. See e.g., Gibson et al., J. Gastroenterol Hepatol. 18:1095-1100 (2003).

Currently, enteritis is mainly managed symptomatically by giving, e.g., conventional antidiarrheal, antiemetic, spasmolytic, and defoaming agents to patients. Somatostatin analogues (e.g., octreotide), 5-HT antagonists, or NK1 antagonists may also be used. See e.g., Keefe et al., Seminars in Oncology 20:38-47 (2004).

Proctitis (mucositis of the rectum) is common is patients who receive radiation therapy for pelvic tumors. Proctitis may be acute radiation proctitis or chronic radiation proctitis. The main symptoms of acute radiation proctitis are diarrhea, tenesmus (fecal urgency with cramp-like rectal pain), and hematochezia (bloody stools). Many patients also experience symptoms from the upper abdomen even though the small bowel is not included in the radiation field. The main symptoms of chronic radiation proctitis are frequent or clustered bowel movements, anal discharge, rectal pain, urgency, tenesmus, incontinence, and hematochezia. See e.g., Keefe et al., Seminars in Oncology 20:38-47 (2004). Topical anesthetic preparations, loperamide, steroid-containing suppositories may be helpful in amolieorating some symptoms. Steroids, 5-aminosalicylic acid, sucralfate enemas, local (endoscopic) intervention with topical formalin, electrocautery, laser therapy, argon plasma beam coagulation, and surgery can be used to manage radiation proctitis with bleeding. Hyperbaric oxygen treatment may be considered in patients with severe symptoms of rectal wall fibrosis or intractable pain caused by rectal ulcers. See e.g., Keefe et al., Seminars in Oncology 20:38-47 (2004).

In summary, there is a great clinical need for methods or agents that can prevent or treat alimentary mucositis effectively. Such methods or agents will be especially beneficial to cancer patients that are receiving or going to receive radiation therapy, chemotherapy, or a combination thereof, and to people who are exposed or going to be exposed to radiation.

### 2.2 Fibroblast Growth Factors

The fibroblast growth factor (“FGF”) family has more than 20 members, each containing a conserved amino acid core (see, e.g., Powers et al., Endocr. Relat. Cancer, 7(3):65-197 (2000)). FGFs regulate diverse cellular functions such as growth, survival, apoptosis, motility, and differentiation (see, e.g., Szebenyi et al., Int. Rev. Cytol., 185:45-106 (1999)). Members of the FGF family are involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7(3):165-197 (2000); and Szebenyi et al., Int. Rev. Cytol. 185:45-106 (1999)).

FGFs transduce signals via high affinity interactions with cell surface tyrosine kinase FGF receptors (FGFRs). These FGF receptors are expressed on most types of cells in tissue culture. For example, FGF receptor-1 (FGFR-1), which shows the broadest expression pattern of the four known FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs may serve to stabilize FGF/FGFR interactions, and to sequester FGFs and protect them from degradation (Szebenyi and Fallon, Int. Rev. Cytol. 185:45-106. (1999)).

Glia-activating factor (“GAF”), another FGF family member, is a heparin-binding growth factor that was purified from the culture supernatant of a human glioma cell line. See, Miyamoto et al., Mol. Cell Biol. 13(7): 4251-4259 (1993). GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N-terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N-terminus with respect to those predicted by the cDNA sequence, except the initiation methionine. The rat FGF-9 cDNA was also cloned, and the structural analysis indicated that the FGF-9 gene is highly conserved.

Through a homology-based genomic mining process, a novel human FGF, FGF-20, was discovered. See U.S. patent application Ser. No. 09/494,585, filed Jan. 13, 2000, and Ser. No. 09/609,543, filed Jul. 3, 2000, the disclosure of each references is incorporated herein by reference. The amino acid sequence of FGF-20 shows close homology with human FGF-9 (70% identity) and FGF-16 (64% identity).

Citation or discussion of a reference herein shall not be construed as an admission that such is prior art to the present invention.

### 3. SUMMARY OF THE INVENTION

The present invention provides methods of preventing and/or treating alimentary mucositis comprising administering to a subject in need thereof a composition comprising one or more CG53135 proteins.

In one embodiment, the present invention provides methods of preventing and/or treating alimentary mucositis comprising administering to a subject in need thereof a prophylactically and/or therapeutically effective amount of an isolated protein selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity.

In another embodiment, the present invention provides methods of preventing and/or treating alimentary mucositis comprising administering to a subject in need thereof a prophylactically or therapeutically effective amount of a protein isolated from a cultured host cell containing an isolated nucleic acid molecule selected from the group consisting of: (a) a nucleic acid molecule comprising a nucleotide sequence selected from the group consisting of SEQ ID NOs: 1, 3, 5, 6, 8, 9, 21, 23, 25, 27, 29, 31, 33, 35, 37, 39 and 41; (b) a nucleic acid molecule encoding a protein comprising an amino acid sequence selected from the group consisting of SEQ ID NOs: 2, 4, 7, 10, 22, 24, 26, 28, 30, 32, 34, 36, 38, or 40; and (c) a nucleic acid molecule hybridizes under stringent conditions to a nucleotide sequence of SEQ ID NO: 1, 3, 5, 6, 8, 9, 21, 23, 25, 27, 29, 31, 33, 35, 37, 39 or 41, or a complement of said nucleic acid molecule. In a specific embodiment, the stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In some specific embodiments, one or more CG53135 proteins are isolated from a cultured eukaryotic cell. In some other specific embodiments, one or more CG53135 proteins are isolated from a cultured prokaryotic cell. In a preferred embodiment, one or more CG53135 proteins are isolated from E. coli. In a specific embodiment, one or more CG53135 proteins isolated from a cultured host cell has a purity of at least 90%, at least 95%, at least 96%, at least 97%, at least 98%, or at least 99%.

In another embodiment, the present invention provides methods of preventing and/or treating alimentary mucositis comprising administering to a subject in need thereof a prophylactically or therapeutically effective amount of a composition comprising a pharmaceutically acceptable carrier, and an isolated protein selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity.

In a specific embodiment, a pharmaceutically acceptable carrier used in accordance to the present invention comprises 0.04M sodium acetate, 3% Glycerol (volume/volume), and 0.2M Arginine-HCl at pH 5.3.

In another specific embodiment, a pharmaceutically acceptable carrier used in accordance to the present invention comprises 0.1-1 M arginine or a salt thereof, 0.01-0.1 M sodium phosphate monobasic (NaH2PO4.H2O), and 0.01% -0.1% weight/volume (“w/v”) polysorbate 80 or polysorbate 20. In one embodiment, the arginine or a salt thereof used in a pharmaceutically acceptable carrier of the invention is selected from the group consisting of arginine, arginine sulfate, arginine phosphate, and arginine hydrochloride. In a preferred embodiment, the arginine or a salt thereof in a pharmaceutically acceptable carrier is of 0.5 M. In another embodiment, sodium phosphate monobasic used in a pharmaceutically acceptable carrier in accordance to the present invention is 0.05 M. In another embodiment, the polysorbate 80 or polysorbate 20 in a pharmaceutically acceptable carrier used in accordance to the present invention is 0.01% (w/v).

In some embodiments, the compositions of the present invention comprising a pharmaceutically acceptable carrier and one or more CG53135 proteins at a concentration of 0.005-50 mg/ml, 0.5-30 mg/ml, 1-30 mg/ml, or 1-10 mg/ml. In a specific embodiment, the compositions of the present invention comprise an isolated protein comprising an amino acid sequence of SEQ ID NO:24. In another specific embodiment, the compositions of the present invention comprise an isolated protein comprising an amino acid sequence of SEQ ID NO:2. In some embodiment, the compositions of the present invention comprise two or more CG53135 proteins. In one embodiment, the compositions of the present invention comprise a first protein comprising an amino acid sequence of SEQ ID NO:24, and a second protein comprising an amino acid sequence of SEQ ID NO:2. In another embodiment, the compositions of the present invention comprise a first protein comprising an amino acid sequence of SEQ ID NO:24, a second protein comprising an amino acid sequence of SEQ ID NO:2, and one or more isolated protein comprising an amino acid sequence selected from the group consisting of SEQ ID NOs: 26, 28, 30 and 32. In yet another embodiment, the compositions of the present invention comprise a first protein comprising an amino acid sequence of SEQ ID NO:24, a second protein comprising an amino acid sequence of SEQ ID NO:2, a third protein comprising an amino acid sequence of SEQ ID NO:28, a fourth protein comprising an amino acid sequence of SEQ ID NO:30, and a fifth protein comprising an amino acid sequence of SEQ ID NO:32. In one embodiment, the compositions of the invention are lyophilized.

In accordance to the present invention, alimentary mucositis that can be prevented or treated include, but is not limited to, oral mucositis, enteritis, esophagitis, stomatitis, and proctitis. Such alimentary mucositis may be caused by a chemical insult, a biological insult, radiation, or a combination thereof.

In one embodiment, the effective amount to prevent and/or treat alimentary mucositis is between 0.001-3 mg/kg, 0.01-1 mg/kg, or 0.01-0.5 mg/kg (protein concentration measured by UV assay). In another embodiment, the effective amount to prevent and/or treat alimentary mucositis is about 0.03 mg/kg, about 0.1 mg/kg, about 0.2 mg/kg, about 0.5 mg/kg, about 1 mg/kg, about 2 mg/kg, or about 3 mg/kg ((protein concentration measured by UV assay). In some embodiments, a composition comprising one or more CG53135 proteins is administered to a subject in need thereof as a single dose at a dosage of 0.001-1 mg/kg, 0.01-0.5 mg/kg, 0.01-0.2 mg/kg, 0.03 mg/kg, 0.1 mg/kg, or 0.2 mg/kg (protein concentration measured by UV assay). In some embodiments, a composition comprising one or more CG53135 proteins is administered to a subject in need thereof as a multiple dosing with each unit dosage of 0.001-0.5 mg/kg, 0.01-0.2 mg/kg, 0.03 mg/kg, 0.1 mg/kg, or 0.2 mg/kg. In some embodiments, a composition comprising one or more CG53135 proteins is administered to a subject in need thereof parenterally (e.g., intravenous administration or subcutaneous administration). In some embodiments, a composition comprising one or more CG53135 proteins is administered to a subject in need thereof by rectal administration, transdermal administration, or transmucosal administration (e.g., nasal administration).

### 3.1 Terminology

As used herein, the term “CG53135”, refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20 (see U.S. Patent Application No. 09/494,585); CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20); CG53135-12 (SEQ ID NOs:21 and 22) represent a single nucleotide polymorphism (“SNP”) of FGF-20 where one amino acid in CG53135-12 is different from SEQ ID NO:2 (the aspartic acid at position 206 is changed to asparagine, “²⁰⁶D→N”). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1 shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic acid molecules that can hybridize to FGF-20 under stringent hybridization conditions.

TABLE 1
Summary of some of the CG53135 family members

| | SEQ ID NO | |
|---|---|---|
| Name | (DNA/Protein) | Brief Description |
| CG53135-01 | 1 and 2 | FGF-20 wild type, stop codon removed |
| CG53135-02 | 3 and 4 | Codon optimized, amino acids 2-54 (as numbered in SEQ ID NO: 2) |
| | | were removed |
| CG53135-03 | 5 and 2 | FGF-20 wild type |
| CG53135-04 | 6 and 7 | Amino acids 20-51 (as numbered in SEQ ID NO: 2) were removed, |
| | | also valine at position 85 is changed to alanine (“⁸⁵V→A”) |
| CG53135-05 | 8 and 2 | Codon optimized, full length FGF-20 |
| CG53135-06 | 9 and 10 | Amino acids 20-51 (as numbered in SEQ ID NO: 2) were removed |
| CG53135-07 | 11 and 12 | Protein consisting of amino acids 1-18 (as numbered in SEQ ID |
| | | NO: 2) |
| CG53135-08 | 13 and 14 | Protein consisting of amino acids 32-52 (as numbered in SEQ ID |
| | | NO: 2) |
| CG53135-09 | 15 and 16 | Protein consisting of amino acids 173-183 (as numbered in SEQ ID |
| | | NO: 2) |
| CG53135-10 | 17 and 18 | Protein consisting of amino acids 192-211 (as numbered in SEQ ID |
| | | NO: 2) |
| CG53135-11 | 19 and 20 | Protein consisting of amino acids 121-137 (as numbered in SEQ ID |
| | | NO: 2) |
| CG53135-12 | 21 and 22 | FGF-20 SNP, aspartic acid at position 206 is changed to asparagines |
| | | (“²⁰⁶D→N”) as compared to CG53135-01 |
| CG53135-13 | 23 and 24 | CG53135-05 minus first 2 amino acids at the N-terminus |
| CG53135-14 | 25 and 26 | CG53135-05 minus first 8 amino acids at the N-terminus |
| CG53135-15 | 27 and 28 | CG53135-05 minus first 11 amino acids at the N-terminus |
| CG53135-16 | 29 and 30 | CG53135-05 minus first 14 amino acids at the N-terminus |
| CG53135-17 | 31 and 32 | CG53135-05 minus first 23 amino acids at the N-terminus |
| IFC 250059629 | 33 and 34 | In frame clone, open reading frame comprising a nucleotide |
| | | sequence encoding amino acids 63-196 of FGF-20 (SEQ ID NO: 2) |
| IFC 250059669 | 35 and 36 | In frame clone, open reading frame comprising a nucleotide |
| | | sequence encoding amino acids 63-211 of FGF-20 (SEQ ID NO: 2) |
| IFC 317459553 | 37 and 38 | In frame clone, open reading frame comprising a nucleotide |
| | | sequence encoding amino acids 63-194 of FGF-20 (SEQ ID NO: 2) |
| | | with ¹⁵⁹G→E |
| IFC 317459571 | 39 and 40 | In frame clone, open reading frame comprising a nucleotide |
| | | sequence encoding amino acids 63-194 of FGF-20 (SEQ ID NO: 2) |
| IFC 250059596 | 41 and 10 | In frame clone, open reading frame comprising a nucleotide |
| | | sequence encoding amino acids 1-19 and 52-211 of FGF-20 (SEQ |
| | | ID NO: 2) |
| IFC 316351224 | 41 and 10 | In frame clone, open reading frame comprising a nucleotide |
| | | sequence encoding amino acids 1-19 and 52-211 of FGF-20 (SEQ |
| | | ID NO: 2). |

As used herein, the term “effective amount” refers to the amount of a therapy (e.g., a composition comprising a CG53135 protein) which is sufficient to reduce and/or ameliorate the severity and/or duration of alimentary mucositis or one or more symptoms thereof, prevent the advancement of alimentary mucositis, cause regression of alimentary mucositis, prevent the recurrence, development, or onset of one or more symptoms associated with alimentary mucositis, or enhance or improve the prophylactic or therapeutic effect(s) of another therapy.

As used herein, the term “FGF-20” refers to a protein comprising an amino acid sequence of SEQ ID NO:2, or a nucleic acid sequence encoding such a protein or the complementary strand thereof.

used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6×sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the terms “prevent,” “preventing,” and “prevention” refer to the prevention of the recurrence, onset, or development of alimentary mucositis or one or more symptoms thereof in a subject resulting from the administration of a therapy (e.g., a composition comprising a CG53135 protein), or the administration of a combination of therapies.

As used herein, the term “prophylactically effective amount” refers to the amount of a therapy (e.g., a composition comprising a CG53135 protein) which is sufficient to result in the prevention of the development, recurrence, or onset of alimentary mucositis or one or more symptoms thereof, or to enhance or improve the prophylactic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult that may induce alimentary mucositis (such as radiation, chemotherapy, or chemical warfare agents). In another embodiment, the subject is a farm animal (e.g., a horse, pig, or cow) or a pet (e.g., a dog or cat) that has been exposed to or is going to be exposed to a similar insult. The term “subject” is used interchangeably with “patient” in the present invention.

As used herein, the terms “treat,” “treatment,” and “treating” refer to the reduction of the progression, severity, and/or duration of alimentary mucositis or amelioration of one or more symptoms thereof, wherein such reduction and/or amelioration result from the administration of one or more therapies (e.g., a composition comprising a CG53135 protein).

As used herein, the term “therapeutically effective amount” refers to the amount of a therapy (e.g., a composition comprising a CG53135 protein), which is sufficient to reduce the severity of alimentary mucositis, reduce the duration of alimentary mucositis, prevent the advancement of alimentary mucositis, cause regression of alimentary mucositis, ameliorate one or more symptoms associated with alimentary mucositis, or enhance or improve the therapeutic effect(s) of another therapy.

### 4. BRIEF DESCRIPTION OF THE DRAWINGS

FIG. 1. RP-HPLC analysis of CG53135-05 E. coli purified product (by Process 1 and 2, respectively, see Section 6.16.1 and 6.16.2 for description of the processes).

FIG. 2. Tryptic map of CG53135-05 E. coli purified product (by Process 1 and 2, respectively).

FIG. 3. Dose Response of CG53135-induced DNA synthesis in NIH 3T3 Fibroblasts. Serum starved NIH 3T3 cells were treated with purified CG53135-01 (CG53135 in figure), 10% serum or vehicle only (control). DNA synthesis was measured in triplicate for each sample, using a BrdU incorporation assay. Data points represent average BrdU incorporation and bars represent standard error (SE).

FIG. 4. CG53135 stimulates Growth of NIH 3T3 Fibroblasts. Duplicate wells of serum starved NIH 3T3 cells were treated for 1 day with purified CG53135-01 (1 ug) or vehicle control. Cell counts for each well were determined in duplicate. Y-axis identifies cell number, which is the average of 4 cell counts (treatment duplicates×duplicate counts) and standard error (SE).

FIG. 5. CG53135 induces DNA synthesis in 786-O Kidney Epithelial cells. Serum starved 786-O cells were left untreated or treated with partially purified CG53135-01 (from 5 ng/uL stock), or with vehicle control (mock). DNA synthesis was measured in triplicate for each sample, using a BrdU incorporation assay. Data points represent average BrdU incorporation and bars represent standard error (SE).

FIG. 6. Effect of CG53135 E. coli purified product in the treatment of radiation-induced mucositis. The total number of days in which animals in each group exhibited a mucositis score >3 was summed and expressed as a percentage of the total number of days scored. Statistical significance of observed differences with the respective vehicle control was calculated using chi-square analysis.

FIG. 7. Effect of Mucositis on the duration of mucositis induced by chemotherapy. The number of days with mucositis scores >3 was evaluated. To examine the levels of clinically significant mucositis as defined by presentation with open ulcers (score>3), the total number of days in which an animal exhibited an elevated score was summed and expressed as a percentage of the total number of days scored for each group. Statistical significance of observed differences was calculated using Chi-square analysis. Vehicle control=disease control.

FIG. 8 shows the cell positions in the crypt.

FIG. 9 shows the crypt survival curve comparing prophylactic administration of CG53135-05 E. coli purified product treatment to PBS control group following different radiation dosages.

FIG. 10 shows the effect of prophylactic administration of CG53135-05 E. coli purified product on mice intestinal crypt survival after radiation insult.

FIGS. 11(A) and (B) show the mean daily mocositis scores following treatment with CG53135-05 E. coli purified product. Mean group mucositis scores were obtained. Error bars represent the standard error of the means (SEM). A comparison of the untreated control group and the groups that received CG53135-05 12 mg/kg IP on days 1 and 2, with the groups that received CG53135-05 on day −1 only was performed. (A) Groups that received CG53135-05 at 6 mg/kg or 12 mg/kg; and (B) Groups that received CG53135-05 at 24 mg/kg or 48 mg/kg.

FIG. 12 shows mean daily mucositis scores following treatment with CG53135-05 E. coli purified product once, twice, thrice or four times. Mean group mucositis scores were obtained. Error bars represent the standard error of the means (SEM). A comparison of the untreated and vehicle control groups with the groups that received CG53135-05 E. coli purified product 12 mg/kg IP was performed. (A) Groups that received CG53135-05 E. coli purified product for one or two days; (B) Groups that received CG53135-05 purified product for three or four days.

FIG. 13 shows percent weight gain in animals with mucositis treated with CG53135-05 purified product. Animals were weighed daily, the percent weight change from day −4 was calculated, and group means and standard errors of the mean (SEM) calculated for each day. A comparison of the untreated control group and the groups receiving CG53135-05 E. coli purified product 12 mg/kg IP on days 1 and 2, with the groups receiving CG53135-05 E. coli purified product on day −1 only was performed. (A) Groups that received CG53135-05 E. coli purified product at 6 mg/kg or 12 mg/kg; (B) Groups that received CG53135-05 E. coli purified product at 24 mg/kg or 48 mg/kg.

FIG. 14 shows Weight change represented as Area Under the Curve (AUC) gain in animals with mucositis treated with CG53135-05 E. coli purified product. The area under the curve (AUC) was calculated for the percent weight change exhibited by each animal in the study. This calculation was made using the trapezoidal rule transformation. Group means were calculated and are shown with error bars representing SEM for each group. A One Way ANOVA was performed to compare groups.

FIG. 15 shows weight change represented as Area Under the Curve (AUC) for animals treated with single dose of CG53135-05 E. coli purified product for one, two, three or four days. The area under the curve (AUC) was calculated for the percent weight change exhibited by each animal in the study. This calculation was made using the trapezoidal rule transformation. Group means were calculated and are shown with error bars representing SEM for each group. A One Way ANOVA was performed to compare groups.

FIG. 16 shows duration of severe mucositis following treatment with CG53135-05 E. coli purified product. Number of days with mucositis scores of 3 or higher. To examine the levels of clinically significant mucositis, as defined by presentation with open ulcers (score >3), the total number of days in which an animal exhibited an elevated score was summed and expressed as a percentage of the total number of days scored for each group. Statistical significance of observed differences was calculated using chi-square analysis. Asterisk (*) denotes significant difference from control.

FIGS. 17(A) and (B) show effects of CG53135 on body weight in animals with gastrointestinal injury induced by whole body irradiation as analyzed by one-way ANOVA and Dunnett's Multiple Comparison Test, respectively.

FIGS. 18(A) and (B) show effects of CG53135 on diarrhea score in mice with gastrointestinal injury induced by whole body irradiation as analyzed by one-way ANOVA and Tukey's Multiple Comparison Test, respectively.

FIG. 19 shows analysis of diarrhea score for each day of observation.

### 5. DETAILED DESCRIPTION OF THE INVENTION

The present invention provides methods for the prevention and/or treatment of alimentary mucositis. In particular, the present invention provides Fibroblast Growth Factor (FGF) 20, its variants, derivatives, homologs, and analogs (collectively referred to as “CG53135”) that can be used in the treatment and/or prevention of alimentary mucositis. While not bound by any theory, the invention is based, in part, on Applicants' discovery that CG53135 has stimulatory effects on the proliferation of epithelial cells and mesenchymal cells, as well as stimulatory effects on stem cells, such as but are not limited to, intestinal stem cells and hematopoietic stem cells, and therefore is effective in preventing and/or treating alimentary mucositis.

For clarity of disclosure, and not by way of limitation, the detailed description of the invention is divided into the following subsections:
(i) CG53135
(ii) Methods of Preparing CG53135
(iii) Characterization and Demonstration of CG53135 Activities and Monitoring Effects During Treatment
(iv) Prophylactic and Therapeutic Uses
(v) Dosage Regimens
(vi) Pharmaceutical Compositions

### 5.1 CG53135

The present invention provides for compositions comprising CG53135 for prevention and/or treatment of alimentary mucositis. As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. In one embodiment, a CG53135 is CG53135-12 (SEQ ID NOs:21 and 22), which is a single nucleotide polymorphism (“SNP”) of FGF-20 (i.e., ²⁰⁶D→N). (For more detailed description of CG53135-12, see e.g., U.S. patent application Ser. No. 10/702,126, filed Nov. 4, 2003, the disclosure of which is incorporated herein by reference in its entirety.) Other examples of SNPs of FGF-20 are also described in U.S. patent application Ser. No. 10/435,087, the content of which is incorporated herein by reference.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

In another embodiment, the invention provides a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein fragment includes, but is not limited to, residues 2-211, 3-211, 9-211, 12-211, 15-211, 24-211, 54-211, or 55-211of FGF-20 (SEQ ID NO:2). In one embodiment, the nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 which are altered by the substitution of different codons that encode a functionally equivalent amino acid residue within the sequence, thus producing a silent change. In a preferred embodiment, a wild-type FGF-20 nucleic acid sequence is codon optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations which result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.® linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2- groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has improved solubility and/or an extended half-life in vivo using any methods known in the art. For example, Fc fragment of human IgG, or inert polymer molecules such as high molecular weight polyethyleneglycol (PEG) can be attached to a CG53135 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

### 5.2 Methods of Preparing CG53135

Methods of isolating a CG53135 protein are described in previous applications, e.g., U.S. patent application Ser. No. 09/609,543, filed Jul. 3, 2000, the content of which is incorporated herein by reference. Any techniques known in the art can be used in purifying a CG53135 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays. The purity of CG53135 can be assayed by any methods known in the art, such as but not limited to, gel electrophoresis. See Scopes, supra. In some embodiment, the CG53135 proteins employed in a composition of the invention can be in the range of 80 to 100 percent of purity, or at least 80%, at least 85%, at least 90%, at least 95%, or at least 98% of purity. In one embodiment, one or more CG53135 proteins employed in a composition of the invention has a purity of at least 99%. In another embodiment, CG53135 is purified to apparent homogeneity, as assayed, e.g., by sodium dodecyl sulfate polyacrylamide gel electrophoresis.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions that enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions that are necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalovirus for effective expression of a CG53135 sequence (Foecking et al., 1986, Gene 45:101; and Cockett et al., 1990, Bio/Technology 8:2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD- protein. These recombinant proteins may be directed into periplasmic space for correct folding and maturation. The fused part can be used for affinity purification of the expressed protein. Presence of cleavage sites for specific proteases like enterokinase allows one to cleave off the CG53135 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region El or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. Expression in a bacterial or yeast system can be used if post-translational modifications are found to be non-essential for a desired activity of CG53135. In a preferred embodiment, E. coli is used to express a CG53135 sequence.

For long-term, high-yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11 (5):l55-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. NatI. Acad. Sci. USA 78:2072); and hygro, which confers resistance to hygromycin (Santerre et al., 1984, Gene 30:147). In addition, mutant cell lines including, but not limited to, tk-, hgprt- or aprt- cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 protein is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135 proteins can be found in Section 6, infra.

### 5.3 Characterization and Demonstration of CG53135 Activities and Monitoring Effects During Treatment

Any methods known in the art can be used to determine the identity of a purified CG53135 protein in a composition used in accordance to the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG53135 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure

The purity of a CG53135 protein in a composition used in accordance to the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG53135 protein in a composition used in accordance to the instant invention is at least 97%, at least 98%, or at least 99% pure by densitometry. In another preferred embodiment, a CG53135 protein in a composition used in accordance to the instant invention is more than 97%, more than 98%, or more than 99% pure by densitometry.

The biological activities and/or potency of CG53135 used in accordance with the present invention can be determined by any methods known in the art. For example, compositions for use in therapy in accordance to the methods of the present invention can be tested in suitable cell lines for one or more activities that FGF-20 possesses (e.g., cellular proliferation stimulatory activity). Non-limiting examples of such assays are described in Section 6.4, infra.

Compositions for use in a therapy in accordance to the methods of the present invention can also be tested in suitable animal model systems prior to testing in humans. Such animal model systems include, but are not limited to, mucositis models in rats, mice, hamsters, chicken, cows, monkeys, rabbits, etc. The principle animal models for mucositis known in the art include, but are not limited to, mice oral mucositis model, Xu et al., Radiother Oncol 1:369-374 (1984); hamster oral mucositis model, Sonis, In: Teicher B (ed) Tumor models in cancer research, Humana Press, Totowa, N.J. (2002); rat gastrointestinal mucositis model, Gibson et al., J Gastroenterol Hepato 18:1095-1100 (2003); mouse intestinal stem cells, Potten et al., Gut 36(6):864-873 (1995).

To establish an estimate of drug activity in mucositis model experiments, an index can be developed that combines observational examination of the animals as well as their survival status. Non-limiting examples are given in Section 6.5, infra. Any staging/scoring system for human patients known in the art may also be used, for example, World Health Organization (WHO) oral mucositis (OM) scoring system an/or the Oral Mucositis Assessment Scale (OMAS) may be used to evaluate the effectiveness of the compositions of the invention in preventing and/or treating oral mucositis.

Further, any assays known to those skilled in the art can be used to evaluate the prophylactic and/or therapeutic utilities of the combinatorial therapies disclosed herein for prevention and/or treatment of alimentary mucositis.

The effectiveness of CG53135 on preventing and/or treating alimentary mucositis can be monitored by any methods known to one skilled in the art, including but not limited to, clinical evaluation, and measuring the level of CG53135 biomarkers in a biosample. CG53135 biomarkers include, but are not limited to, CXCL1, IL-6, and IL-8.

Any adverse effects during the use of CG53135 alone or in combination with another therapy (e.g., another therapeutic or prophylactic agent) are preferably also monitored. Examples of adverse effects of administering a CG53135 protein include, but are not limited to, nausea; chills; fever; vomiting; dizziness; photopsia (vision—“lights flashing”) and astigmatism (mild astigmatism); neuropathy (on soles of the feet); tachycardia; headache; and asymptomatic, and single premature atrial complex noted on ECG. Examples of adverse effects of chemotherapy during a cancer treatment include, but are not limited to, gastrointestinal toxicity such as, but not limited to, early and late forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, as well as constipation, nerve and muscle effects, temporary or permanent damage to kidneys and bladder, flu-like symptoms, fluid retention, and temporary or permanent infertility. Adverse effects from radiation therapy include, but are not limited to, fatigue, dry mouth, and loss of appetite. Other adverse effects include gastrointestinal toxicity such as, but not limited to, early and late forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure. Adverse effects from biological therapies/immunotherapies include, but are not limited to, rashes or swellings at the site of administration, flu-like symptoms such as fever, chills and fatigue, digestive tract problems and allergic reactions. Adverse effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (58th ed., 2004).

### 5.4 Prophylatic and Therapeutic Uses

The present invention provides methods of preventing and/or treating alimentary mucositis comprising administering to a subject in need thereof an effective amount of a composition comprising one or more isolated CG53135 proteins.

Alimentary mucositis that can be prevented and/or treated by the methods of the invention includes, but is not limited to, oral mucositis, esophagitis, stomatitis, enteritis, and proctitis. In some embodiments, the methods of the invention comprise administering an effective amount of a composition comprising one or more isolated CG53135 proteins to a subject with mucositis at more than one area in the alimentary canal (e.g., a subject with both oral mucositis and enteritis). In some embodiments, the methods of the invention comprise administering an effective amount of a composition comprising one or more isolated CG53135 proteins to a subject with mucositis at only one area in the alimentary canal (e.g., a subject with only oral mucositis, or a subject with only enteritis). In a preferred embodiment, the alimentary mucositis that can be prevented and/or treated by the methods of the invention is oral mucositis. In some embodiments, the alimentary mucositis that can be prevented and/or treated by the methods of the invention is not an oral mucositis. Alimentary mucositis may be induced by, e.g., chemical insult, radiation insult, biological insult (e.g., bacteria), or a combination thereof.

The present invention provides methods of preventing and/or treating alimentary mucositis in patient populations with alimentary mucositis and populations at risk to develop alimentary mucositis. In one embodiment, the present invention provides methods of preventing and/or treating alimentary mucositis in a subject who has been treated with radiation therapy and/or chemotherapy. In another embodiment, the present invention provides methods of preventing alimentary mucositis by administering a composition comprising one or more CG53135 proteins to a subject who is going to be treated with radiation therapy and/or chemotherapy. In a specific embodiment, the present invention provides methods of preventing and/or treating alimentary mucositis in a subject who has been treated with conditioning myeloablative radiation therapy and/or chemotherapy in preparation for autologous or allogenic hematopoietic stem cell transplant. In another specific embodiment, the present invention provides methods of preventing and/or treating alimentary mucositis in a subject who has received or is receiving mucosatoxic chemotherapy with mucositis-inducing agents (e.g., leukemia patients treated with cytarabine). In yet another specific embodiment, the present invention provides methods of preventing and/or treating alimentary mucositis in a subject who has head and/or neck cancer treated with radiation therapy with or without adjuvant chemotherapy.

In one embodiment, the present invention provides a method of preventing alimentary mucositis comprising administering a composition comprising one or more CG53135 proteins prior to an insult (e.g., a chemical insult, a radiation insult, a biological insult, or a combination thereof) that may induce alimentary mucositis occurs to a subject. In another embodiment, the present invention provides a method of preventing alimentary mucositis comprising administering a composition comprising one or more CG53135 proteins after an insult (e.g., a chemical insult, a radiation insult, a biological insult, or a combination thereof) that may induce alimentary mucositis occurs to a subject, but prior to the development of alimentary mucositis in the subject. In yet another embodiment, the present invention provides a method of treating alimentary mucositis comprising administering a composition comprising one or more CG53135 proteins after alimentary mucositis developed in a subject.

In some embodiments, the present invention provides a method of preventing and/or treating alimentary mucositis comprising cyclically administering a composition comprising one or more CG53135 proteins. In one embodiment, cycling therapy involves the administration of a first therapy for a period of time, followed by the administration of a second therapy for a period of time and repeating this sequential administration, i.e., the cycle, in order to, e.g., to avoid or reduce the side effects of one of the therapies and/or to improve the efficacy of the therapies. In another embodiment, cycling therapy involves the administration of a therapy for a period of time, stop the therapy for a period of time, and repeat the administration of the therapy. In accordance to the present invention, a composition comprising one or more CG53135 proteins can be administered to a subject prior to, during, or after the administration of a radiation therapy and/or chemotherapy, where such radiation therapy and/or chemotherapy is a cycling therapy.

In accordance to the instant invention, a composition comprising one or more isolated CG53135 proteins can also be used in combination with other therapies to prevent and/or treat alimentary mucositis. In one embodiment, a composition comprising one or more isolated CG53135 proteins is administered in combination with one or more other agents that have prophylactic and/or therapeutic effect(s) on alimentary mucositis and/or have amelioration effect(s) on one or more symptoms associated with alimentary mucositis to a subject to prevent and/or treat alimentary mucositis. Non-limiting examples of such agents are: mucosal protective agents (e,g, sucralfate, colloidal bismuth), antibiotics, antifungal agents (e.g., fluconazole, amphotericin B), antiviral agents (e.g., acyclovir), antiemetic agents (e.g., phenothiazines, butyrophenones, benzodiazepines, corticosteroids, cannabinoids, 5-HT3 serotonin receptor blockers), antidiarrhea agents (e.g., diphenoxylate, loperamide, kaolin, pectin, methylacellulose, activated attapulgite, magnesium aluminum silicate, non-steroidal anti-inflammatory agents (NSAIDs)), transforming growth factor (TGF), interleukin-11 (IL-11), granulocyte-macrophage colony stimulating factor (GM-CSF), keratinocyte growth factor (KGF), L-glutamine, Amifostene, and Granulocyte colony stimulating factor (G-CSF). In another embodiment, a composition comprising one or more isolated CG53135 proteins is administered in combination with one or more other therapies that have palliative effect on alimentary mucositis. Non-limiting examples of such therapies are: application of topical analgesics such as lidocaine and/or systemic administration of narcotics and antibiotics, topical fluoride application with or without calcium phosphate, mechanical plaque removal, tooth sponges, sucking ice chips resulting in oral cooling, oral rinses with various anti-infective agents, oral mouthwashes with local anesthetics.

### 5.5 Dosage Regimens

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more G53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg (as measured by UV assay). In another embodiment, the dosage of a composition comprising one or more CG53135 proteins for administration in a human patient provided by the present invention is between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

In one embodiment, prior to administering the first full dose, each patient preferably receives a subcutaneous injection of a small amount (e.g., 1/100 to 1/10 of the prescribed dose) of a composition of the invention to detect any acute intolerance. The injection site is examined one and two hours after the test. If no reaction is detected, then the full dose is administered.

### 5.6 Pharmaceutical Compositions

The compositions used in accordance to the present invention can be administered to a subject at a prophylactically or therapeutically effective amount to prevent and/or treat alimentary mucositis. Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g.,arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, micelles, emulsions, sphingomyelins, lipid-protein or lipid-peptide complexes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M -0.2 M acetate, 0.5-5% glycerol, 0.2-0.5 M arginine-HCl, and one ore more CG53135 proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH₂PO₄·H₂O), 0.01%-0.1% weight/volume (“w/v”) polysorbate 80 or polysorbate 20, and one or more CG53135 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135 proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLE

Certain embodiments of the invention are illustrated by the following non-limiting examples.

### 6.1 Example 1

### Identification of Single Nucleotide Polymorphisms in FGF-20 Nucleic Acid Sequences

This example demonstrated how some of the single nucleotide polymorphisms (SNPs) of FGF-20 were identified. A SNP can, in some instances, be referred to as a “cSNP” to denote that the nucleotide sequence containing the SNP originates as a cDNA. SNPs occurring within a gene may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Non-limiting examples include alteration in temporal expression, physiological response regulation, cell type expression regulation, intensity of expression, and stability of transcribed message.

SeqCalling™ assemblies produced by the exon linking process were selected and extended using the following criteria: genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling™ assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling™ assemblies map to those regions. Such SeqCalling™ sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling™ database. SeqCalling™ fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling™ assemblies and genomic clones was reiterated to derive the full length sequence (Alderborn et al., Genome Research 10 (8) 1249-1265 (2000)).

Variants are reported individually in Table 2, but any combination of all or select subset of the variants is also encompassed by the present invention.

TABLE 2
SNPs of CG53135-01 (SEQ ID NOs: 1 and 2)

| | Nucleotides | | | Amino Acids | | |
|---|---|---|---|---|---|---|
| Variant | Position | Initial | Modified | Position | Initial | Modified |
| 13377871 | 301 | A | G | 101 | Ile | Val |
| 13375519 | 361 | A | G | 121 | Met | Val |
| 13375518 | 517 | G | A | 173 | Gly | Arg |
| 13375516 | 523 | C | G | 175 | Pro | Ala |
| 13381791 | 616 | G | A | 206 | Asp | Asn |

### 6.2 Example 2

### Expression of CG53135

Several different expression constructs were generated to express CG53135 proteins (Table 3). The CG53135-05 construct, a codon-optimized, phage-free construct encoding the full-length gene (construct #3 in Table 3), was expressed in E. coli BLR (DE3), and the purified protein product was used in toxicology studies and clinical trials.

TABLE 3
Constructs Generated to Express CG53135

| Construct | Construct Description | Construct Diagram |
|---|---|---|
| 1a | NIH 3T3 cells were transfected with pFGF-20, which incorporates an epitope tag (V5) and a polyhistidine tag into the carboxy-terminus of the CG53135-01 protein in the pcDNA3.1 vector (Invitrogen) | 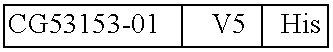 |
| 1b | Human 293-EBNA embryonic kidney cells or NIH 3T3 cells were transfected with CG53135-01 using pcEP4 vector (Invitrogen) containing an IgK signal sequence, multiple cloning sites, a V5 epitope tag, and a polyhistidine tag | 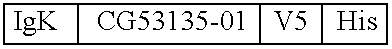 |
| 2 | E. coli BL21 cells were transformed with CG53135-01 using pETMY vector (CuraGen Corporation) containing a polyhistidine tag and a T7 epitope tag (this construct is also referred to as E. coli/pRSET) | 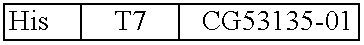 |
| 3 | E. coli BLR (DE3) cells (NovaGen) were transformed with CG53135-05 (full-length, codon-optimized) using pET24a vector (NovaGen) | 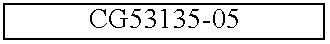 |
| 4 | E. coli BLR (DE3) cells (NovaGen) were transformed with CG53135 (deletion of amino acids 2-54, codon-optimized) using pET24a vector (NovaGen) | 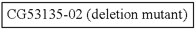 |

In one construct, CG53135-01 (the full-length CG53135 gene) was cloned as a Bgl II-Xho I fragment into the Bam HI-Xho I sites in mammalian expression vector, pcDNA3.1V5His (Invitrogen Corporation, Carlsbad, Calif.). The resultant construct, pFGF-20 (construct 1a) has a 9 amino acid V5 tag and a 6 amino acid histidine tag (His) fused in-frame to the carboxy-terminus of CG53135-01. These tags aid in the purification and detection of CG53135-01 protein. After transfection of pFGF-20 into murine NIH 3T3 cells, CG53135-01 protein was detected in the conditioned medium using an anti-V5 antibody (Invitrogen, Carlsbad, Calif.).

The full-length CG53135-01 gene was also cloned as a Bgl II-Xho I fragment into the Bam HI-Xho I sites of mammalian expression vector pCEP4/Sec (CuraGen Corporation). The resultant construct, plgK-FGF-20 (construct 1b) has a heterologous immunoglobulin kappa (IgK) signal sequence that could aid in secretion of CG53135-01. After transfection of plgK-FGF-20 into human 293 EBNA cells (Invitrogen, Carlsbad, Calif.; catalog #R620-07), CG53135-01 was detected in the conditioned medium using an anti-V5 antibody.

In order to increase the yield of CG53135 protein, a Bgl II-Xho I fragment encoding the full-length CG53135-01 gene was cloned into the Bam HI-Xho I sites of E. coli expression vector, pETMY (CuraGen Corporation). The resultant construct, pETMY-FGF-20 (construct 2) has a 6 amino acid histidine tag and a T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express CG53135 without tags, CG53135-05 (a codon-optimized, full-length FGF-20 gene) and CG53135-02 (a codon-optimized deletion construct of FGF-20, with the N-terminal amino acids 2-54 removed) were synthesized. For the full-length construct (CG53135-05), an Nde I restriction site (CATATG) containing the initiator codon was placed at the 5′ end of the coding sequence. At the 3′ end, the coding sequence was followed by 2 consecutive stop codons (TAA) and a Xho restriction site (CTCGAG). The synthesized gene was cloned into pCRScript (Stratagene, La. Jolla, Calif.) to generate pCRScript-CG53135. An Nde I-Xho I fragment containing the codon-optimized CG53135 gene was isolated from the pCRscript-CG53135 and subcloned into Nde l-Xho I-digested pET24a to generate pET24a-CG53135 (construct 3). The full-length, codon-optimized version of CG53135 is referred to as CG53135-05.

To generate a codon-optimized deletion construct for CG53135, oligonucleotide primers were designed to amplify the deleted CG53135 gene from pCRScript-CG53135. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde I-Hind III fragment was isolated from pCR2.1-53135del and subcloned into Nde I-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside. Both the full-length and the N-terminally truncated CG53135 protein was detected in the soluble fraction of cells.

### 6.3 Example 3

### Proteolytic Cleavage Products of CG53135-05

When pET24a-CG53135-05 (construct 3, see Example 2) was expressed in E. coli (DE3) and the protein was purified according to Process 1 as described in Section 6.16.1 and Process 2 as described in Section 6.16.2, respectively, the final purified protein product from each process was analyzed using techniques such as Liquid Chromatography, Mass spectrometry and N-terminal sequencing. Such analyses indicate that the final purified protein product includes some truncated form of FGF-20 (e.g., CG53135-13 (SEQ ID NO:24), CG53135-15 (SEQ ID NO:28), CG53135-16 (SEQ ID NO:30), and CG53135-17 (SEQ ID NO:32)) in addition to the full length FGF-20, and a protein consisting of amino acids 3-211 (CG53135-13, SEQ ID NO:24) of FGF-20 constitutes the majority of the final purified protein product.

All the variants/fragments in the final purified product have high activity in the proliferation assays. Thus these variants/fragments are expected to have same utility as that of FGF-20. For the purpose of convenience, the term “CG53135-05 E. coli purified product” is used herein to refer to a purified protein product from E. coli expressing a CG53135-05 construct. For example, a CG53135-05 E. coli purified product may contain a mixture of the full length CG53135-05 protein (SEQ ID NO:2), CG53135-13 (SEQ ID NO:24), CG53135-15 (SEQ ID NO:28), CG53135-16 (SEQ ID NO:30), and CG53135-17 (SEQ ID NO:32), with the majority of the content being CG53135-13 (SEQ ID NO:24).

RP-HPLC Assay: Peak Identification

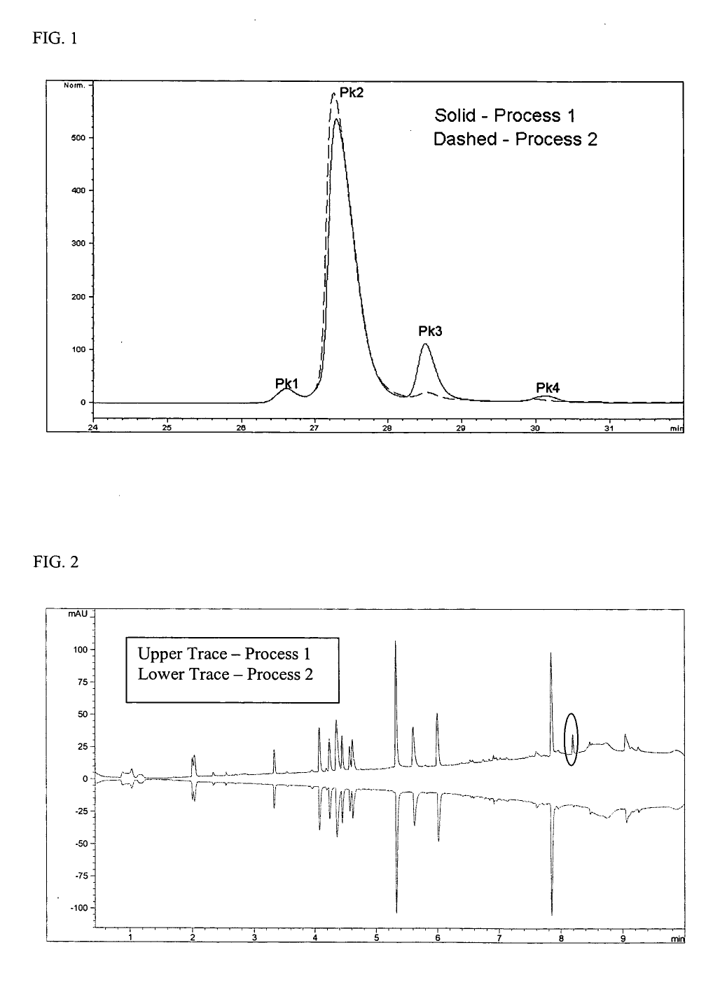

Purified drug substance (by both Process 1 and Process 2, respectively) was further analyzed by reversed-phase high-performance liquid chromatography (RP-HPLC) with both UV and electrospray mass spectrometric detection. Purified protein from either Process 1 or Process 2 was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 1). These peaks were characterized by electrospray mass spectrometry. As can be observed from the chromatograms, the four equipotent isoforms are present in the purified final product from Process 1 and 2. However, the proportion of these peaks (1, 3 and 4) is much lower in the final product purified by Process 2 with the predominant form being Peak 2.

The identities of each peak from the RP-HPLC separation are indicated in Table 4.

TABLE 4
Identity of peaks from the RP-HPLC separation of CG53135-05 E. coli
purified product based upon accurate molecular weight determination.

| | | Molecular | | Predicted |
|---|---|---|---|---|
| | Retention | Weight | Assignment | Molecular |
| Peak # | Time (min) | Observed | (residue #) | Weight |
| 1 | 26.6 | 21329.2 | 24-211 | 21329.2 |
| 1 | 26.6 | 22185.1 | 15-211 | 22185.1 |
| 1 | 26.6 | 22412.4 | 12-211 | 22412.4 |
| 2 | 27.3 | 23296.5 | 3-211 | 23296.4 |
| 3 | 28.5 | 23498.9 | 1-211 | 23498.7 |
| 4 | 30.0 | 23339.3 | 3-211(carbamylated) | 23339.4 |
| 4 | 30.0 | 23539.7 | 1-211(carbamylated) | 23539.7 |

Edman Sequencing and Total Amino Acid Analysis

The experimental N-terminal amino acid sequence of the Process 1 reference standard, DEV10, and the Process 2 interim reference standard were determined qualitatively. The reference standards were resolved by SKS-PAGE and electrophoretically transferred to a polyvinylidenefluoride membrane; the Coomassie-stained ˜23 kDa major band corresponding to each reference standard was excised from the membrane and analyzed by an automated Edman sequencer (Procise, Applied Biosystems, Foster City, Calif.). A comparison of the two major sequences is shown in Table 5 below. The predominant sequence for each reference standard was identical and corresponded to residues 3-20 in the theoretical N-terminal sequence of CG53135-05.

TABLE 5
Edman sequencing data for the first 20 amino acids of CG53135-05
E. coli purified product for Process 1 and 2.

| | |
|---|---|
| Theoretical | Amino Acid |
| Residue | Residue |

| Position | Process 1 | Process 2 |
|---|---|---|
| 3 | Pro | Pro |
| 4 | Leu | Leu |
| 5 | Ala | Ala |
| 6 | Glu | Glu |
| 7 | Val | Val |
| 8 | Gly | Gly |
| 9 | Gly | Gly |
| 10 | Phe | Phe |
| 11 | Leu | Leu |
| 12 | Gly | Gly |
| 13 | Gly | Gly |
| 14 | Leu | Leu |
| 15 | Glu | Glu |
| 16 | Gly | Gly |
| 17 | Leu | Leu |
| 18 | Gly | Gly |
| 19 | Gln | Gln |
| 20 | Gln | Gln |

The experimental amino acid composition of the DEV10 reference standard and the PX3536G001-H reference standard were determined in parallel. Quadruplicate samples of each reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μl sample buffer containing 2 nmol homoserine as an internal standard. The amino acids in each sample were separated on a Beckman Model 7300 amino acid analyzer. The amino acid composition of both reference standards showed no significant differences as shown in Table 6 below. Note that Cys and trp are destroyed during acid hydrolysis of the protein. Asn and gin are converted to asp and glu, respectively, during acid hydrolysis and thus their respective totals are reported as asx and glx. Met and his were both unresolved in this procedure.

TABLE 6
Quantitive amino acid analysis for CG53135-05 E. coli
purified product from Process 1 and Process 2

| | |
|---|---|
| Amino Acid | Mole Percent |

| Residue | DEV10 | PX3536G001-H |
|---|---|---|
| asx | 7.1 | 7.0 |
| thr | 4.0 | 4.0 |
| ser | 6.3 | 6.1 |
| glx | 12.2 | 12.2 |
| pro | 6.0 | 6.0 |
| gly | 14.4 | 14.3 |
| ala | 5.8 | 5.6 |
| val | 5.3 | 5.3 |
| ile | 3.5 | 3.5 |
| leu | 13.6 | 13.6 |
| tyr | 4.6 | 4.6 |
| phe | 5.2 | 5.2 |
| lys | 3.7 | 3.7 |
| arg | 8.5 | 9.1 |

Tryptic Mapping by RP-HPLC

Purified drug substance from Process 1 and 2 was reduced and alklated with iodoacetic acid and then digested with sequencing grade trypsin. The tryptic peptides were separated by reversed-phase high-performance liquid chromatography (RP-HPLC) using both UV and electrospray mass spectrometric detection. The tryptic digest from either Process 1 or Process 2 was loaded onto an ODS-1 nonporous silica column (Micra, 1.5 μm; 53×4.6 mm) using a standard HPLC system in a mobile phase containing water, acetonitrile and trifluoroacetic acid. The eluting peptides were detected by UV at 214 nm (FIG. 2) and by positive-ion electrospray mass spectrometry. The major difference between the two chromatograms for Process 1 and Process 2 is the reduction in peak area of a peak obvious in the Process I trace (peak at 8.2 min; FIG. 2). This peak corresponds to the T1 peptide, residues 1-40. This observation is expected since the source of this peptide if from the intact CG53135-05, which is in greater abundance in the Process 1 material (peak 3, FIG. 1).

Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 7). The PI₂₀₀ was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 7).

TABLE 7
Biological Activity of CG53135-05 E. coli purified product
(DEV10): Induction of DNA Synthesis

| | CG53135-05 | | | |
|---|---|---|---|---|
| | (DEV 10) | Peak 1 | Peak 2 | Peak 3 |
| PI₂₀₀ (ng/mL) | 1.0 | 0.7 | 11 | 8.6 |

### 6.4 Example 4

### Cellular Proliferation Responses with CG53135 (Studies L-117.01 and L-117.02)

Experiments were performed to evaluate the proliferative response of representative cell types to CG53135, e.g., a full-length tagged variant (CG53135-01), a deletion variant (CG53135-02), and a full-length codon-optimized untagged variant (CG53135-05).

Materials and Methods:

Heterologous Protein Expression: CG53135-01 (batch 4A and 6) was used in these experiments. Protein was expressed using Escherichia coli (E. coli ), BL21 (Novagen, Madison, Wis.), transformed with full-length CG53135-01 in a pETMY-hFGF20X/BL21 expression vector. Cells were harvested and disrupted, and then the soluble protein fraction was clarified by filtration and passed through a metal chelation column. The final protein fraction was dialyzed against phosphate buffered saline (PBS) plus 1 M L-arginine. Protein samples were stored at −70° C.

CG53135-02 (batch 1 and 13) was also used in these experiments. Protein was expressed in E. coli, BLR (DE3) (Novagen), transformed with the deletion variant CG53135-02 inserted into a pET24a vector (Novagen). A research cell bank (RCB) was produced and cell paste containing CG53135-02 was produced by fermentation of cells originating from the RCB. Cell membranes were disrupted by high-pressure homogenization, and lysate was clarified by centrifugation. CG53135-02 was purified by ion exchange chromatography. The final protein fraction was dialyzed against the formulation buffer (100 mM citrate, 1 mM ethylenediaminetetraacetic acid (EDTA), and 1 M L-arginine).

CG53135-05, DEV10, which were also used in these experiments, was prepared by Cambrex Biosciences (Hopkinton, Mass.) according to Process 1 as described in Section 6.16.1, infra.

BrdU Incorporation: proliferative activity was measured by treatment of serum-starved cultured cells with a given agent and measurement of BrdU incorporation during DNA synthesis. Cells were cultured in respective manufacturer recommended basal growth medium supplemented with 10% fetal bovine serum or 10% calf serum as per manufacturer recommendations. Cells were grown in 96-well plates to confluence at 37° C. in 10% CO₂/air (to subconfluence at 5% CO₂ for dedifferentiated chondrocytes and NHOst). Cells were then starved in respective basal growth medium for 24-72 hours. CG53135 protein purified from E. coli or pCEP4/Sec or pCEP4/Sec-FGF 20X enriched conditioned medium was added (10 μl/100 μL of culture) for 18 hours. BrdU (10 μM final concentration) was then added and incubated with the cells for 5 hours. BrdU incorporation was assayed according to the manufacturer's specifications (Roche Molecular Biochemicals, Indianapolis, Ind.).

Growth Assay: growth activity was obtained by measuring cell number following treatment of cultured cells with a given agent for a specified period of time. In general, cells grown to ˜20% confluency in 6-well dishes were treated with basal medium supplemented with CG53135 or control, incubated for several days, trypsinized and counted using a Coulter Z1 Particle Counter.

Results:

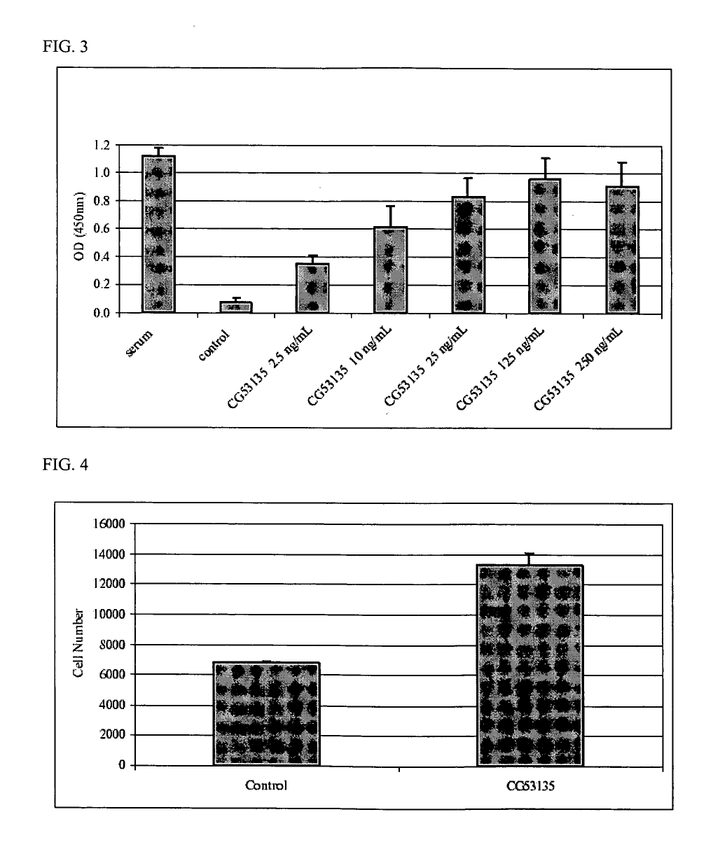

Proliferation in Mesenchymal Cells: to determine if recombinant CG53135 could stimulate DNA synthesis in fibroblasts, a BrdU incorporation assay was performed using CG53135-01 treated NIH 3T3 murine embryonic lung fibroblasts. Recombinant CG53135-01 induced DNA synthesis in NIH 3T3 mouse fibroblasts in a dose-dependent manner (FIG. 3). DNA synthesis was generally induced at a half maximal concentration of ˜10 ng/mL. In contrast, treatment with vehicle control purified from cells did not induce any DNA synthesis.

CG53135-01 also induced DNA synthesis in other cells of mesenchymal origin, including CCD-1070Sk normal human foreskin fibroblasts, MG-63 osteosarcoma cell line, and rabbit synoviocyte cell line, HIG-82. In contrast, CG53135-01 did not induce any significant increase in DNA synthesis in primary human osteoblasts (NHOst), human pulmonary artery smooth muscle cells, human coronary artery smooth muscle cells, human aorta smooth muscle cells (HSMC), or in mouse skeletal muscle cells.

To determine if recombinant CG53135-01 sustained cell growth, NIH 3T3 cells were cultured with 1 μg CG53135-01 or control for 48 hours and then counted (FIG. 4). CG53135 induced an approximately 2-fold increase in cell number relative to control in this assay. These results show that CG53135 acts as a growth factor.

Proliferation of Epithelial Cells: to determine if recombinant CG53135 can stimulate DNA synthesis and sustain cell growth in epithelial cells, a BrdU incorporation assay was performed in representative epithelial cell lines treated with CG53135. Cell counts following protein treatment were also determined for some cell lines.

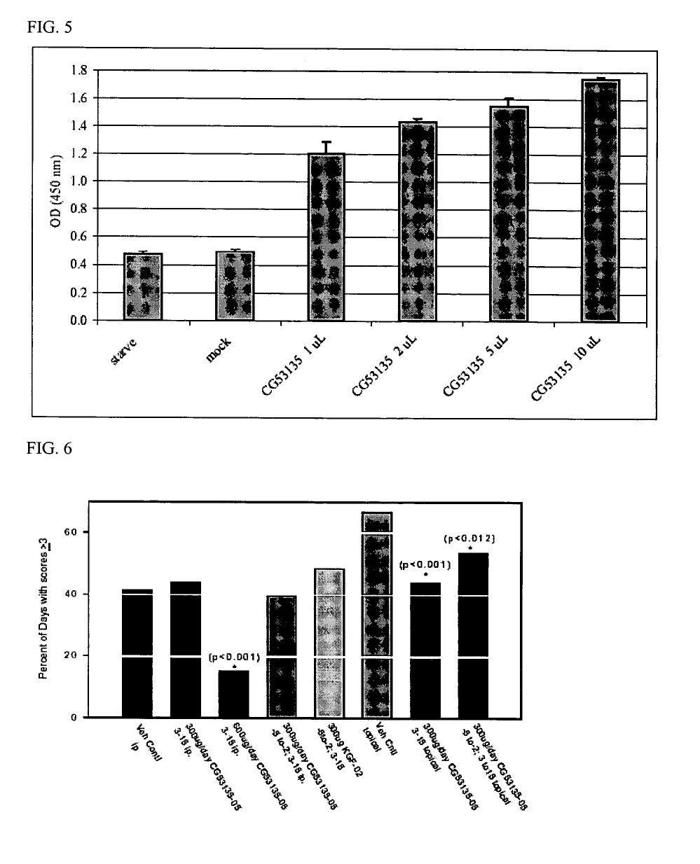

CG53135 was found to induce DNA synthesis in the 786-O human renal carcinoma cell line in a dose-dependent manner (FIG. 5). In addition, CG53135-01 induced DNA synthesis in other cells of epithelial origin, including CCD 1106 KERTr human keratinocytes, Balb MK mouse keratinocytes, and breast epithelial cell line, B5589.

Proliferation of Hematopoietic Cells: no stimulatory effect on DNA synthesis was observed upon treatment of TF-1, an erythroblastic leukemia cell line with CG53135-01. These data suggest that CG53135-01 does not induce proliferation in cells of erythroid origin. In addition, Jurkat, an acute T-lymphoblastic leukemia cell line, did not show any response when treated with CG53135-01, whereas a robust stimulation of BrdU incorporation was observed with serum treatment.

Effects of CG53135 on Endothelial Cells: protein therapeutic agents may inhibit or promote angiogenesis, the process through which endothelial cells differentiate into capillaries. Because CG53135 belongs to the fibroblast growth factor family, some members of which have angiogenic properties, the antiangiogenic or pro-angiogenic effects of CG53135 on endothelial cell lines were evaluated. The following cell lines were chosen because they are cell types used in understanding angiogenesis in cancer: HUVEC (human umbilical vein endothelial cells), BAEC (bovine aortic endothelial cells), HMVEC-d (human endothelial, dermal capillary). These endothelial cell types undergo morphogenic differentiation and are representative of large vessel (HUVEC, BAEC) as well as capillary endothelial cells (HMVEC-d).

CG53135-01 treatment did not alter cell survival or have stimulatory effects on BrdU incorporation in human umbilical vein endothelial cells, human dermal microvascular endothelial cells or bovine aortic endothelial cells. Furthermore, CG53135-01 treatment did not inhibit tube formation, an important event in formation of new blood vessels, in HUVECS. This result suggests that CG53135 does not have anti-angiogenic properties. Finally, CG53135-01 had no effect on VEGF induced cell migration in HUVECs, suggesting that it does no play a role in metastasis.

The above described experiments were also performed using CG53135-02 and CG53135-05 protein products, and the results are summarized in the Conclusion section below.

Conclusions

Recombinant CG53135-01 (which encode the same protein as CG53135-05) induces a proliferative response in mesenchymal and epithelial cells in vitro (i.e., NIH 3T3 mouse fibroblasts, CCD-1070 normal human skin fibroblasts, CCD-1106 human keratinocytes, 786-O human renal carcinoma cells, MG-63 human osteosarcoma cells and human breast epithelial cells), but not in human smooth muscle, erythroid, or endothelial cells. Like CG53135-01 and CG53135-05, CG53135-02 also induces proliferation of mesenchymal and epithelial cells. In addition, CG53135-02 induces proliferation of endothelial cells.

### 6.5 Example 5

### Activity of CG53135 in Hamster Model of Acute Radiation-Induced Oral Mucositis (N-152 Study)

CG53135 protein was evaluated for activity in a hamster model of radiation-induced oral mucositis, and its activity compared with KGF-2, another FGF family member. KGF-2, also referred to as FGF-10, is active in models of wound healing and inflammatory bowel disease (Miceli et al. J. Pharmacol. Exp. Ther. 290:464-471 (1999)). Protein concentrations in this example were measured by Bradford assay.

The acute radiation model in hamsters (Sonis et al., Oral Surg Oral Med Oral Pathol 69:437-443 (1990)) has proven to be an accurate, efficient, and cost-effective technique to provide a preliminary evaluation of anti-mucositis compounds, including growth factors and cytokines (Sonis et al., Oral Oncol 36:373-381 (2000); Sonis et al., Cytokine 9:605-612 (1997); Sonis et al., Oral Oncol 33:47-54 (1997)). The acute model has little systemic toxicity, resulting in few animal deaths, permitting the use of smaller groups for initial activity studies. It has also been used to study specific mechanistic elements in the pathogenesis of mucositis. Molecules that show activity in the acute radiation model may be further evaluated in the more complex models of fractionated radiation, chemotherapy, or concomitant therapy. In this model, an acute radiation dose of approximately 40Gy on Day 0 is administered in order to induce severe mucositis. This dose results in predictable ulcerative oral mucositis that typically peaks around Day 16-18.

Materials and Methods:

CG53135-05 protein used in this study was purified as Batch Dev 08-02. The recombinant human DNA protein, CG53135-05, was expressed using Escherichia coli BLR (DE3) cells (Novagen, Darmstadt, Germany). These cells were transformed with full-length, codon-optimized CG53135-05 using pET24a vector (Novagen). A GMP manufacturing cell bank (MCB) of these cells was produced. Cell paste containing CG53135-05 protein, produced by fermentation of cells originating from the MCB, was lysed with high pressure homogenization in lysis buffer, and clarified by centrifugation. CG53135-05 was purified from clarified cell lysate by 2 cycles of ion exchange chromatography and ammonium sulfate precipitation. The final precipitate was washed with purified water and suspended in formulation buffer as follows: 30 mM citrate (pH 6.0), 2 mM EDTA, 200 mM sorbitol, 50 mM KCl, 20% glycerin.

Male Golden Syrian hamsters (Charles River Laboratories or Harlan), of age 6 to 7 weeks, and with similar body weight (mean body weight 77.4 g) in all groups at study commencement, were used in this study. Sixty-four hamsters were randomized into 8 groups of 8 animals each prior to irradiation. Each group was assigned a different treatment as shown in Table 8.

TABLE 8
Treatment Groups

| Group | No. of | | | Volume (mL); |
|---|---|---|---|---|
| No. | Animals | Treatment | Treatment Days | Treatment |
| 1 | 8 males | vehicle control IP | Days −5 to −2; 3 to 15 | 0.1; once/day |
| 2 | 8 males | 300 μg/day CG53135-05 E. coli | Days 3 to 15 | 0.1; once/day |
| | | purified product IP | | |
| 3 | 8 males | 600 μg/day CG53135-05 E. coli | Days 3 to 15 | 0.1; once/day |
| | | purified product IP | | |
| 4 | 8 males | 300 μg/day CG53135-05 E. coli | Days −5 to −2; 3 to 15 | 0.1; once/day |
| | | purified product IP | | |
| 5 | 8 males | 300 μg/day KGF-2 IP | Days −5 to −2; 3 to 15 | 0.125; once/day |
| 6 | 8 males | vehicle control topical | Days −5 to −2; 3 to 15 | 0.2; three times/day |
| 7 | 8 males | 300 μg/day CG53135-05 E. coli | Days 3 to 15 | 0.2; three times/day |
| | | purified product topical | | |
| 8 | 8 males | 300 μg/day CG53135-05 E. coli | Days −5 to −2; 3 to 15 | 0.2; three times/day |
| | | purified product topical | | |

Animals were acutely radiated with a single dose of radiation (40Gy/dose) on the left buccal mucosa on Day 0. Animals were treated once daily with vehicle or CG53135-05 E. coli purified product intraperitoneally (IP) or topically following acute radiation. Animals in Groups 1 to 5 received IP injection of test materials once per day. For Groups 6 to 8, test material was applied topically to the cheek pouch three times per day. The following dosing schedules were used: Day 3 to Day 15 (Groups 2, 3 and 7), and Day−5 (i.e., five days prior to radiation) to Day−2 (i.e., two days prior to radiation), then Day 3 to Day 15 (Groups 1, 4, 5, 6 and 8). Doses of CG53135-05 E. coli purified product were 300 μg/day (Groups 2, 4, 7 and 8) and 600 μg/day (Group 3). The KGF-2 dose was 300 μg/day (Group 5). Mucositis was evaluated on alternate days beginning on Day 6 and continued until the conclusion of the experiment on Day 28 (i.e., Days 6, 8, 10, 12, 14, 16, 18, 20, 22, 24, 26 & 28). Clinically relevant oral mucositis (e.g., mucositis score of≧3) developed ˜14 days after radiation.

Each hamster was weighed daily for the period of the study (i.e., Day−5 to Day 28) and its survival was recorded in order to assess possible differences in animal weight among treatment groups as an indication for mucositis severity or possible toxicity resulting from the treatments. Mucositis was scored visually by comparison to a validated photographic scale, ranging from 0 (normal) to 5 (for severe ulceration). The clinical scale is described in Table 9.

TABLE 9
Mucositis Scoring Definitions

| Score: | Description: |
|---|---|
| 0 | Pouch completely healthy. No erythema or vasodilation. |
| 1 | Light to severe erythema and vasodilation. No erosion of mucosa |
| 2 | Severe erythema and vasodilation. Erosion of superficial aspects |
| | of mucosa leaving denuded areas. Decreased |
| | stippling of mucosa. |
| 3 | Formation of off-white ulcers in one or more places. Ulcers may |
| | have a yellow/gray due to pseudomembrane. Cumulative size of |
| | ulcers should equal about ¼ of the pouch. |
| | Severe erythema and vasodilation. |
| 4 | Cumulative size of ulcers should equal about ½ of the pouch. |
| | Loss of pliability. Severe erythema and vasodilation. |
| 5 | Virtually all of pouch is ulcerated. Loss of pliability (pouch |
| | can only partially be extracted from mouth). |

A score of 1-2 is considered to represent a mild stage of the disease, whereas a score of 3-5 is considered to indicate moderate to severe mucositis. Following clinical scoring, a photograph was taken of each animal's mucosa using a standardized technique. At the conclusion of the experiment, all film was developed and the photographs randomly numbered for blinded scoring. Thereafter, 2 independent, trained observers graded the photographs in blinded fashion using the above-described scale. For each photograph the actual blinded score was be based upon the average of the score assigned by the 2 blinded, independent evaluators. Only the scores from blinded photographic evaluation was statistically analyzed and reported in the results.

The effect of each treatment on mucositis compared to the vehicle control group was assessed according to the parameters listed in Table 10.

TABLE 10
Parameters for Evaluation of Activity

| Parameter | Description |
|---|---|
| The difference in the number | On each evaluation day, the number of |
| of days hamsters in each | animals with a blinded mucositis score |
| group have severe mucositis | of ≧3 in each drug treatment group was |
| (score ≧3). | compared to the vehicle control group. |
| | Differences were analyzed on a |
| | cumulative basis. Treatment success |
| | was considered a statistically significant |
| | lower number of hamsters with this score |
| | in a drug treatment group, versus the |
| | vehicle control value, as determined by |
| | chi-square analysis. |
| The rank sum differences in | For each evaluation day the scores of the |
| daily mucositis scores. | vehicle control group was compared to |
| | those of the treated group using the non- |
| | parametric rank sum analysis. Treatment |
| | success was considered as a statistically |
| | significant lowering of scores in the |
| | treated group on 2 or more days from |
| | Day 6 to Day 28. |

Results

There were no statistically significant differences in survival or weight change over time between the two vehicle control groups and their respective test groups.

Prophylactic treatment with either 300 μg/animal/day CG53135-05 E. coli purified product or KGF-2, administered IP prior to and after radiation (Day−5 to Day−2 then Day 3 to Day 15) failed to elicit significant activity in reducing the incidence of moderate to severe mucositis (FIG. 6). Treatment with 300 μg/animal/day CG53135-05 E. coli purified product administered IP from Day 3 to Day 15 also failed to elicit significant activity in reducing the incidence of moderate to severe mucositis (FIG. 6).

Treatment with 600 μg/animal/day CG53135-05 E. coli purified product administered IP from Day 3 to Day 15 showed only one day of significant activity by rank sum analysis (p<0.001) (FIG. 6). Though treatment success criteria for this analysis have been defined as two or more days of significant activity, this observation suggests that this treatment has a favorable effect on mucositis. This group also had a statistically significant lower score than corresponding control treatment by chi square analysis (p<0.001). Therefore, this combination of dose, schedule and route of administration is active in treating mucositis in this model.

Treatment with 300 μg/animal/day CG53135-05 E. coli purified product administered topically from Day 3 to Day 15 showed significant activity in reducing the incidence of moderate to severe mucositis by Chi square analysis (p<0.001). Treatment was also considered successful by rank sum analysis as mucositis was significantly reduced on five of the twelve scoring days. Therefore, this combination of dose, schedule and route of administration of CG53135-05 E. coli purified product has activity in treating mucositis in this model. Treatment success criteria by chi square analysis were met (p=0.012) when 300 μg/animal/day CG53135-05 E. coli purified product was administered topically prior to and after radiation (i.e., Day −5 to Day −2 and Day 3 to Day 15). Therefore, this combination of dose, schedule and route of administration of CG53135-05 E. coli purified product showed activity in reducing the incidence of moderate to severe mucositis.

In an additional experiment, IP treatment with 300 μg/animal/day CG53135-01 (a tagged, full-length form of CG53135), on Day 3 to 15 also had a beneficial effect on the course and severity of mucositis in the acute radiation model of mucositis in golden Syrian hamsters (N-135 study).

In yet another experiment, untreated control and vehicle-injected control animals were compared with animals treated intraperitoneally with 300, 600, or 1200 μg CG53135-05 E. coli purified product (an untagged, full-length form of CG53135 with a slightly different formulation from that used in the experiment above) from Day 3 to Day 15 (N-197 study). No beneficial effect was observed in male animals treated with 300 μg CG53135-05 E. coli purified product. However, consistent with the results reported above, treatment with 600 μg CG53135-05 E. coli purified product resulted in a significant reduction in the severity of mucositis compared with untreated control animals (p<0.001 by Chi-square analysis) and significantly reduced mean daily mucositis scores for 3 of 12 scoring days compared with the vehicle control group. In addition, administration of 1200 μg CG53135-05 E. coli purified product significantly reduced the severity of mucositis relative to the vehicle control group (p<0.001 by Chi-square analysis) and significantly reduced mean daily mucositis scores for 5 scoring days. No significant difference in body weight was observed in any of the treatment regimens when compared with the controls.

Conclusions

The activity of CG53135 was evaluated in a model of oral mucositis induced in hamsters administered a single, bolus dose of radiation (40Gy) on Day 0. Clinically relevant oral mucositis (e.g., mucositis score of≧3) developed ˜14 days after radiation. In general, treatment with CG53135 after radiation insult significantly reduced clinically relevant mucositis. Treatment with CG53135 (3 mg/kg/day topical administration for 18 days or 6-12 mg/kg/day intraperitoneal administration for up to 18 days) reduced the severity of mucositis. No studies were conducted using an intravenous (IV) route of administration since IV administration in hamsters is technically challenging and data are consequently highly variable.

### 6.6 Example 6

### Activity of CG53135 In Hamster Model of Chemotherapy-Induced Oral Mucositis (N-212 Study)

CG53135 was evaluated for the treatment of chemotherapy-induced oral mucositis in male Golden Syrian hamsters (protein concentrations in this Example were measured by Bradford assay).

Materials and Methods

CG53135-05 used in this study (batch 29-NB849:76) was expressed and purified as described in Section 6.5, with the exception that the final protein fraction was dialyzed against formulation buffer containing 30 mM sodium citrate, 2 mM EDTA, 200 mM sorbitol, 50 mM KCl, 20% glycerol (pH 6.1).

Male golden Syrian hamsters (Charles River Laboratories) age 5 to 6 weeks and with similar body weight in all groups at study commencement were used in this study. Sixty male hamsters were randomized into 6 groups of 10 animals each prior to irradiation. The treatment groups are outlined in Table 11.

TABLE 11
Treatment Groups

| Group No. | Treatment (0.1 mL, IP) | Dosing Schedule |
|---|---|---|
| 1 | Vehicle (Disease control) | Day 1 to Day 18 |
| 2 | CG53135-05 E. coli purified product, | Day 1 to Day 18 |
| | 12 mg/kg/day | |
| 3 | CG53135-05 E. coli purified product, | Day 6 to Day 14 |
| | 12 mg/kg/day | |
| 4 | CG53135-05 E. coli purified product, | Day 1 to Day 9 |
| | 12 mg/kg/day | |
| 5 | CG53135-05 E. coli purified product, | Day 1 to Day 6 |
| | 12 mg/kg/day | |
| 6 | CG53135-05 E. coli purified product, | Day 1 to Day 2 |
| | 12 mg/kg/day | |

Mucositis was induced using 5-fluorouracil, delivered as single bolus (60 mg/kg, IP) on Days−4 and −2. A single submucosatoxic dose of radiation (40Gy/dose) was locally administered to all animals on Day 0. Animals were treated once daily with 0.1 mL vehicle or 12 mg/kg CG53135-05 IP following mucosa toxic insult according to the schedule shown in Table 11. Mucositis was scored visually as described in Section 6.5 (Table 9) on alternate days beginning on Day 6 and every second day until the conclusion of the experiment on Day 30 (i.e., Days 8, 10, 12, 14, 16, 18, 20, 22, 24, 26, 28, and 30). Each hamster was weighed daily for the period of the study (i.e., Day 0 to Day 30). Weight and survival were monitored as indices for severity of mucositis or possible toxicity resulting from treatment.

The effect of each treatment on mucositis compared with the control group was assessed according to the parameters listed in Table 12. Statistical differences between treatment groups were determined using the Student's t-test, Mann-Whitney U test, and chi-square analysis, with a critical value of 0.05.

TABLE 12
Parameters for evaluation of Activity

| Parameter | Description |
|---|---|
| The difference in the number | On each evaluation day, the number of |
| of days hamsters in each | animals with a blinded mucositis score |
| group have severe | of ≧3 in each drug treatment group was |
| mucositis (score ≧3). | compared to the vehicle control group. |
| | Differences were analyzed on a |
| | cumulative basis. Treatment success |
| | was considered a statistically significant |
| | lower number of hamsters with this score |
| | in a drug treatment group, versus the |
| | vehicle control value, as determined by |
| | chi-square analysis. |
| The rank sum differences in | For each evaluation day the scores of the |
| daily mucositis scores. | vehicle control group was compared to |
| | those of the treated group using the non- |
| | parametric rank sum analysis. Treatment |
| | success was considered as a statistically |
| | significant lowering of scores in the |
| | treated group on 2 or more days from |
| | Day 6 to Day 30. |

Results

There were no statistically significant differences in weight or survival over time between the vehicle control group (Group 1) and CG53135-05 E. coli purified product treatment groups (Groups 2-6).

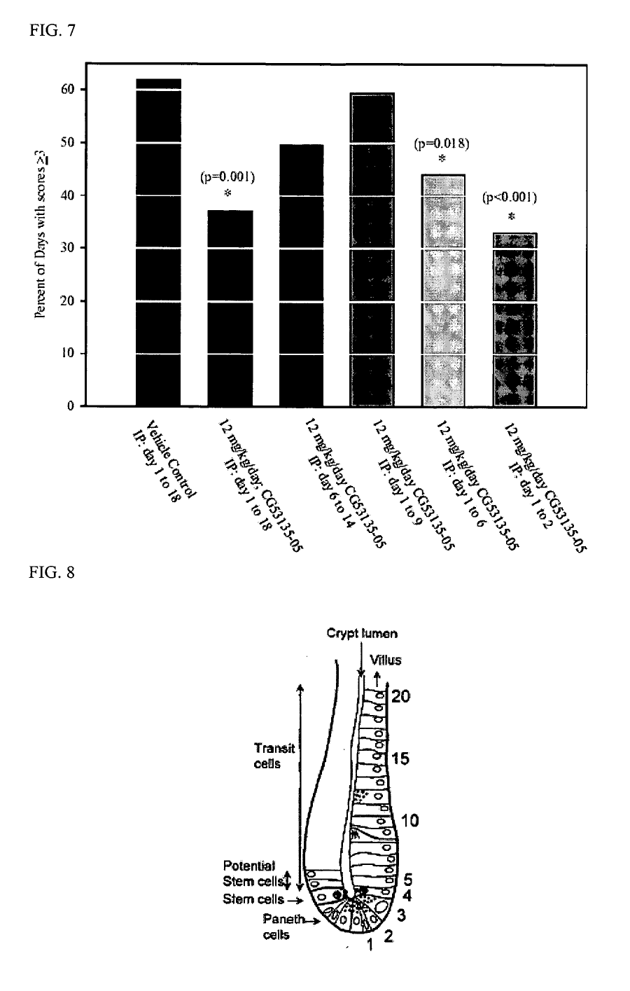

In this model of mucositis primarily induced by chemotherapy, dosing schedule was important in the treatment of oral mucositis. Administration of CG53135-05 E. coli purified product (12 mg/kg/day) from Day 6 to Day 14 or Day 1 to Day 9 did not result in significant improvement in the course or severity of mucositis (FIG. 7). Administration of CG53135-05 E. coli purified product (12 mg/kg/day) from Day 1 to Day 18 or Day 1 to Day 6 resulted in significant improvement of the duration of severe mucositis (Chi-square analysis). However, these treatments did not result in significant improvement of daily mucositis scores (rank sum analysis). Treatment with 12 mg/kg/day CG53135-05 E. coli purified product (Day 1 to Day 2) had a significant effect on both the course and severity of mucositis in this study (FIG. 7). These results suggest that a short-course of treatment with a CG53135-05 E. coli purified product immediately after a combined chemotherapy and radiation regimen improves the outcome of the disease in this model of mucositis.

In another experiment, treatment of hamsters with 12 mg/kg/day CG53135-05 E. coli purified product starting after radiation (Day 1 to Day 18) resulted in a significant reduction of ulceration (p<0.001) combined with 7 days of significant reduction in mucositis scores, as determined by rank sum analysis (N-198 study). This suggests that the administration of a CG53135-05 E. coli purified product results in a significantly beneficial treatment of radiation-induced oral mucositis when administered after mucosa toxic insult.

In yet another experiment, administration of 12 mg/kg/day of CG53135-05 E. coli purified product (formulated in 40 mM sodium acetate, 0.2 M L-arginine, and 3% glycerol) on Days 1 to 2 significantly reduced the severity of mucositis (N-237 study). These results confirm the findings presented above.

Conclusions

The activity of CG53135 was evaluated in a model of mucositis induced in hamsters treated with 60 mg/kg 5-flourouracil on Days −4 and −2, followed by a single sub-mucosatoxic dose of radiation (˜30Gy) on Day 0. Clinically relevant oral mucositis (mucositis score of ≧3) developed ˜Day 15. Intraperitoneal administration of CG53135 for 2, 6, or 18 days significantly reduced severity of mucositis.

### 6.7 Example 7

### Effect of CG53135-05 Administration on Hamster Epithelial Proliferation In Vivo (N-225 STUDY)

The experiment described herein evaluated in vivo incorporation of BrdU into the gastrointestinal epithelium and bone marrow after a single dose of a CG53135-05 E. coli purified product (protein concentrations in this example were measured by Bradford assay).

Materials and Methods

Male Golden Syrian hamsters (Charles River Laboratories or Harlan Sprague Dawley), aged 5 to 6 weeks, with a mean body weight of 82 g at study commencement were used. Twenty-five male hamsters were randomized into 5 groups of 5 animals each as outlined in Table 13.

TABLE 13
Treatment Groups

| Group | No. of | | Euthanasia/ | Volume (mL); |
|---|---|---|---|---|
| No. | Animals | Treatment | Necropsy | Treatment |
| 1 | 5 males | BrdU 50 mg/kg, IP, (0 hrs) | 2 hrs | Adjust by body |
| | | | | weight |
| 2 | 5 males | 12 mg/kg CG53135-05 E. coli purified | 2 hrs | Adjust by body |
| | | product, IP (0 hrs) + BrdU 50 mg/kg, | | weight |
| | | IP, (0 hrs) | | |
| 3 | 5 males | 12 mg/kg CG53135-05 E. coli purified | 4 hrs | Adjust by body |
| | | product, IP (0 hrs) + BrdU 50 mg/kg, | | weight |
| | | IP, (2 hrs) | | |
| 4 | 5 males | 12 mg/kg CG53135-05 E. coli purified | 8 hrs | Adjust by body |
| | | product, IP (0 hrs) + BrdU 50 mg/kg, | | weight |
| | | IP, (6 hrs) | | |
| 5 | 5 males | 12 mg/kg CG53135-05 E. coli purified | 24 hrs | Adjust by body |
| | | product, IP (0 hrs) + BrdU 50 mg/kg, | | weight |
| | | IP, (22 hrs) | | |

A single dose of a CG53135-05 E. coli purified product at 12 mg/kg IP was administered and hamsters were sacrificed at 2, 4, 8 and 24 hours post-administration.

BrdU Administration and Immunohistochemistry: all animals received BrdU 50 mg/kg IP two hours before sacrifice, allowing for uptake of the reagent into proliferating tissues. At euthanasia, the following tissues were harvested: cheek pouch mucosa, esophagus, stomach, duodenum, jejunum, ileum, cecum, colon, rectum and sternum. All tissue samples were fixed in 10% neutral buffered formalin for 24 hours and then transferred to 70% ethanol. Samples were trimmed, paraffin embedded, sectioned and mounted. Epithelial tissues were stained for incorporation of BrdU by immunohistochemistry using Oncogene Research products BrdU Immunohistochemistry kit Catalog # HCS24 in accordance with the manufacturer's instructions.

Results

The effect of CG53135-05 E. coli purified product on the incorporation of BrdU into all tissues was essentially the same: a relatively small increase in the number of BrdU labeled nuclei was observed 2 hours after the administration of CG53135-05 E. coli purified product. This was followed by a decrease in the number of labeled nuclei at 4 hours after the administration of CG53135-05 E. coli purified product. All tissues showed a dramatic increase in BrdU labeling at 8 hours post administration. At 24 hours, all tissues except rectum showed a decrease in the number of labeled nuclei compared with the untreated controls, while the rectal tissue showed a slight increase over the controls. Since no labeled cells were seen in the rectal tissue samples from the untreated animals, the observation of 2 labeled cells in the 24 hour time point has to be regarded as observational error, or data scatter, since there must be a low level of cell replication in the tissue.

Conclusions

The in vivo mechanistic activity of CG53135 was evaluated using bromodexoyuridine labeling in vivo to evaluate the effect of a single bolus dose (12 mg/kg) of CG53135-05 E. coli purified product on mucosal tissue over a 24-hour period. CG53135-05 E. coli purified product stimulated the division of the epithelial cells of the cheek pouch, jejunum and rectum as well as the hematopoietic cells of the bone marrow. Peak increases in BrdU incorporation in these tissues were seen at 8 hours after the administration of CG53135-05. All tissues showed the same time response to the administration of CG53135-05 E. coli purified product.

### 6.8 Example 8

### Modulation of Intestinal Crypt Cell Proliferation and Apoptosis by CG53135-05 Administration to Mice (N-342)

This study evaluated the effect of CG53135 on small intestinal crypt cell turnover in order to discriminate stem cell versus daughter cell effects, and to draw insights regarding the mode of action of CG53135 in syndromes associated with gastrointestinal stem cell damage (e.g., mucositis). Furthermore, the effect of CG53135 on stem cell radiosensitivity was also assessed. Protein concentrations in this example were measured by Bradford assay.

A “crypt” is a hierarchical structure with the stem cells towards the crypt base. As cells become more mature, they move progressively from the bottom of the crypt towards the top of the crypt. Therefore, changes that may be affecting stem cells versus their transit amplifying daughter cells can be detected by looking at changes in event frequency at each cell position. The cell positions are marked in FIG. 8. Thus, the effects of CG53135 on the crypt microarchitecture were analyzed in the context of crypt cellularity.

Experimental Design

Animals were sacrificed at various times after a single 12 mg/kg (IP) dose of a CG53135-05 E. coli purified product. Just prior to sacrifice the mice were labeled with a single injection of bromodeoxyuridine to label S-phase cells and determine the effect of the drug on crypt cell proliferation/apoptosis. Mice were weighed and then dosed with a CG53135-05 E. coli purified product (12 mg/kg, single injection, ip). Groups of 6 animals were sacrificed 0, 3, 6, 9, 12, 24, 48 hours post injection with a CG53135-05 E. coli purified product. All received a single injection of bromodeoxyuridine 40 minutes prior to sacrifice (see Table 14).

An additional two groups of 6 mice were used to assess the effects of CG53135-05 E. coli purified product on stem cell radiosensitivity (groups 8 and 9, see Table 14). One group was treated with a CG53135-05 E. coli purified product (12 mg/kg, single injection, ip) and another group was injected with a placebo control. Twenty-four hours post injection, animals were irradiated with 1Gy X-ray (specifically to induce stem cell apoptosis) followed by routine in vivo BrdU labeling. Animals were sacrificed 4.5 hours later (at time of peak apoptosis).

TABLE 14
Study Design

| | Number | | |
|---|---|---|---|
| Group | of | | Treatment |
| Number | Animals | Treatment | Schedule* |
| 1 | 6 males | CG53135-05 E. coli | Injected and euthanize 3 hr later |
| | | purified product, | 40 mg/kg BrdU 40 min prior to |
| | | 12 mg/kg, IP | sacrifice |
| 2 | 6 males | CG53135-05 E. coli | Injected and euthanize 6 hr later |
| | | purified product, | 40 mg/kg BrdU 40 min prior to |
| | | 12 mg/kg, IP | sacrifice |
| 3 | 6 males | CG53135-05 E. coli | Injected and euthanize 9 hr later |
| | | purified product, | 40 mg/kg BrdU 40 min prior to |
| | | 12 mg/kg, IP | sacrifice |
| 4 | 6 males | CG53135-05 E. coli | Injected and euthanize 12 hr |
| | | purified product, | later 40 mg/kg BrdU 40 min |
| | | 12 mg/kg, IP | prior to sacrifice |
| 5 | 6 males | CG53135-05 E. coli | Injected and euthanize 24 hr |
| | | purified product, | later 40 mg/kg BrdU 40 min |
| | | 12 mg/kg, IP | prior to sacrifice |
| 6 | 6 males | CG53135-05 E. coli | Injected and euthanize 48 hr |
| | | purified product, | later 40 mg/kg BrdU 40 min |
| | | 12 mg/kg, IP | prior to sacrifice |
| 7 | 6 males | Untreated | 40 mg/kg BrdU 40 min prior to |
| | | | sacrifice |
| 8 | 6 males | CG53135-05 E. coli | Dose 24 hr prior to irradiation |
| | | purified product, | Euthanize 4.5 hr post irradiation |
| | | 12 mg/kg, IP | 40 mg/kg BrdU 40 min prior to |
| | | 1 Gy X ray | sacrifice |
| 9 | 6 males | PBS, IP | Dose 24 hr prior to irradiation |
| | | 1 Gy X-ray | Euthanize 4.5 hr post irradiation |
| | | | 40 mg/kg BrdU 40 min prior to |
| | | | sacrifice |

Intestinal Crypt Cell Proliferation and Apoptosis Modulation: Procedure

All S-phase dividing cells incorporate the injected bromodeoxyuridine (BrdU) and hence are marked as cycling cells. Animals that were irradiated were placed, unanaesthetised, in a perspex jig and subjected to whole body radiation of 1Gy X-ray at a dose rate of 0.7Gy/min. This low level of radiation induced apoptosis in the small intestinal stem cell population, but not in the more mature cells.

The small intestine was removed, fixed in Carnoy's fixative, and processed for histological analysis (paraffin embedded). One set of 3 mm sections were immunolabeled for BrdU and one set of sections were stained with H&E. Longitudinal sections of small intestinal crypts were analyzed for the presence of either BrdU or apoptotic/mitotic nuclei. Fifty half crypts were scored per animal.

Groups 1-7 (Group A in the results) were tested to determine the effect of CG53135-05 E. coli purified product over a 48 hour period. Groups 8-9 (Group B in the results) were tested to determine whether CG53135-05 E. coli purified product changes the number of apoptotic cells generated after low dose irradiation, i.e., whether CG53135-05 E. coli purified product influences the radiosensitive stem cell population.

The results generated show a frequency distribution for the crypts in each group of animals that were further analyzed for statistical differences. Tissue samples were harvested at 3, 6, 9, 12, 24, and 48 hours after treatment with CG53135-05 E. coli purified product. Apoptosis, mitotic index, and proliferation were the end points for this study.

Results:

Group A.

In groups 1-7 (Table 14), CG53135-05 E. coli purified product had no significant effect on spontaneous apoptosis. Similar results were obtained with the mitotic index (Table 15). However, results of BrdU uptake as in Table 15, revealed the following:
a) At 3 hour, there was extension/increase of proliferative region (positions 12-22).
b) By 9 hours, large proliferative effects were noted in many positions.
c) By 12 hours, only positions 4-8 showed increase in uptake (stem cells).
d) By 24 hours, there was a significant inhibition of proliferation.

e) By 48 hours, the uptake was comparable to control levels.

TABLE 15
Summary of significant cell positions in the crypt after assessment
of apoptosis, mitosis, and proliferation

| Sample time | Significant Cell Positions | | |
|---|---|---|---|
| (hours) | | Apoptotic | Mitotic |
| After treatment | BrdU labeling Index | Index | Index |
| 3 | 12 to 22 | None | None |
| 6 | None | None | None |
| 9 | 5 to 9 & 11 to 20 to 21 | None | None |
| 12 | 4 to 8 | None | None |
| 24 | 4 to 8 | None | None |
| 48 | None | None | None |

The comparisons shown in Table 15 are between treated groups versus the untreated group. The cell positions shown are the ones that are significantly different from the untreated control (P<0.05).

Group B:

In Groups 8 and 9 (Table 14), stem cell radiosensitivity was assessed. As shown in Table 14, CG53135-05 E. coli purified product or PBS was administered one day before dosing with 1Gy radiation. Tissues were harvested 4.5 hours after radiation dosing. There was no significant effect on both radiation-induced apoptosis and the mitotic index. However, increased uptake in positions 4-8 by 12 hours and significant inhibition of proliferation were seen in mice pretreated with CG53135-05 E. coli purified product and irradiated, consistent with the Group A results (Table 15).

### 6.9 Example 9

### Effect of CG53135-05 Prophylactic Administration on Mice Intestinal Crypt Survival After Radiation Injury (N-343)

The purpose of this study was to evaluate the efficacy of CG53135 against radiation-induced crypt cell mortality in vivo using the Clonoquant™ assay. Protein concentrations in this example were measured by Bradford assay.

Mice were weighed and then dosed with a CG53135-05 E. coli purified product (12 mg/kg) or placebo. A single injection was given, intraperitoneally (ip), 24 hours prior to irradiation. Each group of 6 animals was irradiated as per table below. For each radiation dose, the response of a drug treated group and a placebo treated group was compared.

The small intestine was removed, fixed in Carnoy's fixative, and processed for histological analysis (paraffin embedded). H&E sections were prepared following conventional protocols. For each animal, ten intestinal circumferences were analyzed, the number of surviving crypts per circumference was scored, and the average per group was determined. Only crypts containing 10 or more strongly H&E stained cells (excluding Paneth cells) and only intact circumferences, not containing Peyers patches, were scored.

The average crypt width (measured at its widest point) was also measured in order to correct for scoring errors due to crypt size difference. The correction was applied as follows:

Corrected number of crypts per circumference=Mean number of surviving crypts per circumference in treatment group X (Mean crypt width in untreated control/Mean crypt width in treated animal).

TABLE 16
Study design

| Group | Number of | | | Treatment |
|---|---|---|---|---|
| Number | Animals | Induction | Treatment | Schedule* |
| 1 | 6 males | 10 Gy | PBS | Day - 1 |
| | | Day 0 | | |
| 2 | 6 males | 11 Gy, | PBS | Day - 1 |
| | | Day 0 | | |
| 3 | 6 males | 12 Gy, | PBS | Day - 1 |
| | | Day 0 | | |
| 4 | 6 males | 13 Gy, | PBS | Day - 1 |
| | | Day 0 | | |
| 5 | 6 males | 14 Gy, | PBS | Day - 1 |
| | | Day 0 | | |
| 6 | 6 males | 10 Gy | CG53135-05 E. coli | Day - 1 |
| | | Day 0 | purified | |
| | | | product, | |
| | | | 12 mg/kg, IP | |
| 7 | 6 males | 11 Gy, | CG53135-05 E. coli | Day - 1 |
| | | Day 0 | purified | |
| | | | product, | |
| | | | 12 mg/kg, IP | |
| 8 | 6 males | 12 Gy, | CG53135-05 E. coli | Day - 1 |
| | | Day 0 | purified | |
| | | | product, | |
| | | | 12 mg/kg, IP | |
| 9 | 6 males | 13 Gy, | CG53135-05 E. coli | Day - 1 |
| | | Day 0 | purified | |
| | | | product, | |
| | | | 12 mg/kg, IP | |
| 10 | 6 males | 14 Gy, | CG53135-05 E. coli | Day - 1 |
| | | Day 0 | purified | |
| | | | product, | |
| | | | 12 mg/kg, IP | |
| 11 | 6 males | Untreated | | |

Results:

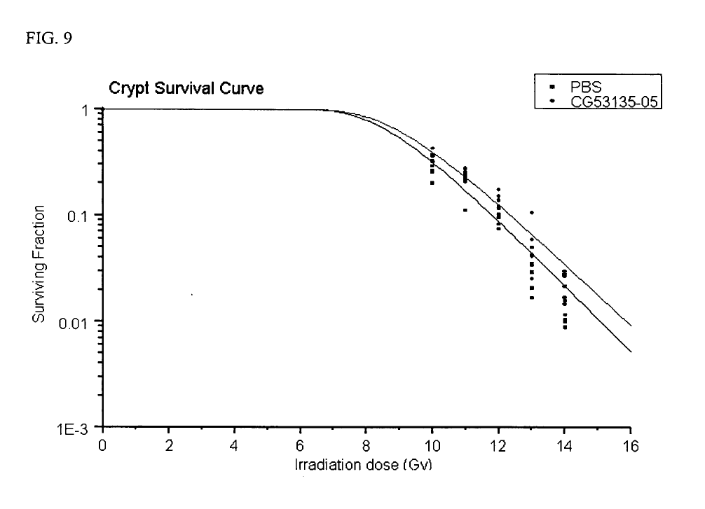

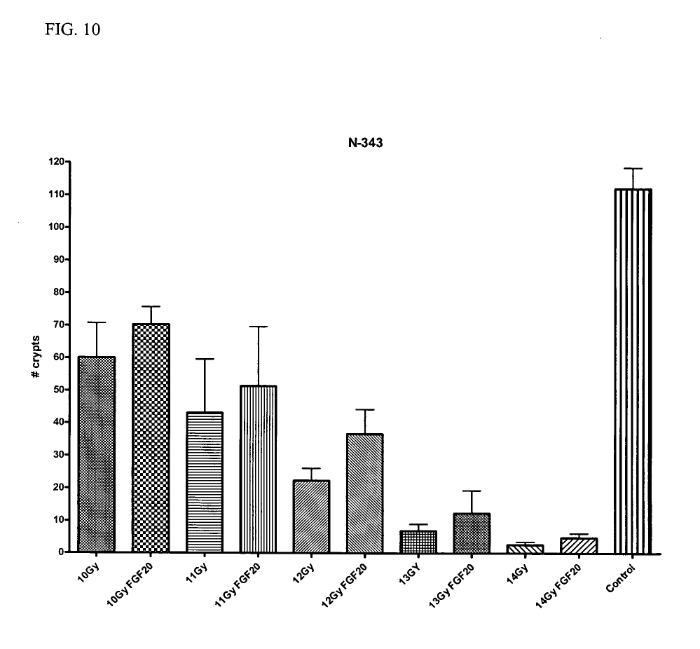

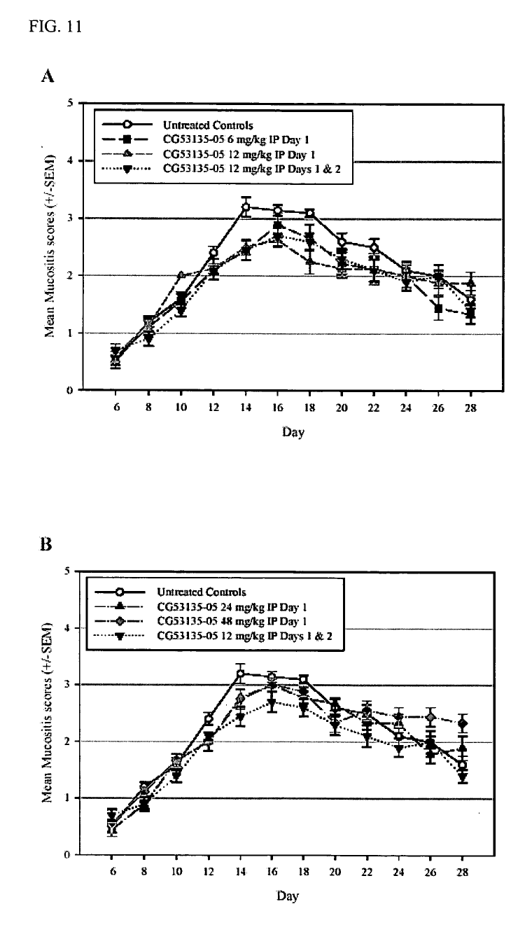

The crypt survival following prophylactic CG53135-05 E. coli purified product administration showed inverse correlation to the irradiation dose, the lesser the irradiation dose, the higher was the crypt survival (FIGS. 9 and 10). Prophylactic administration of CG53135-05 E. coli purified product significantly increased the number of crypts (P<0.001). Table 17 shows the protection factor achieved for the radiation doses following prophylactic administration of the protein (CG53135-05 E. coli purified product). Protection factor (Table 17) represents the ratio between treated and untreated cells. On average, 1.55 times as many cells survived irradiation dose of 12Gy, when animals were administered with CG53135-05 E. coli purified product prior to the radiation insult.

TABLE 17

| Radiation dose (Gy) | Protection Factor |
|---|---|
| 10 | 1.29 |
| 11 | 1.21 |
| 12 | 1.55 |
| 13 | 1.71 |
| 14 | 1.73 |

### 6.10 Example 10

### Effect of CG53135 Administration on Chemotherapy/Radiation Model of Oral Mucositis (N-346 STUDY)

Material and Methods

Escherichia coli BLR (DE3) cells (Novagen, Madison, Wisc.) were transformed with full-length, codon-optimized CG53135-05 using pET24a vector (Novagen), and a manufacturing master cell bank (MMCB) of these cells was produced. Cell paste containing CG53135-05 produced by fermentation of cells originating from the MMCB was lysed with high-pressure homogenization in lysis buffer and clarified by centrifugation. CG53135-05 was purified from clarified cell lysate by 2 cycles of ion exchange chromatography and ammonium sulfate precipitation. The final protein fraction was dialyzed against the formulation buffer (30 mM citrate, pH 6.0, 2 mM ethylenediaminetetraacetic acid (EDTA), 200 mM sorbitol, 50 mM KCl, 20% glycerol). Vehicle contains 30 mM sodium citrate, pH 6.1, 2 mM EDTA, 200 mM sorbitol, 50 mM KCl, 20% glycerol. Protein concentrations in this example were measured by Bradford assay.

Golden Syrian hamsters (Charles River Laboratories or Harlan), of age 5 to 6 weeks, and with an average body weight of 84 g at study commencement, were used in this study. Animals were individually numbered using an ear punch and housed in small groups of up to 7 animals per cage. Animals were acclimated prior to study commencement. During this period, the animals were observed daily in order to reject animals in poor condition.

Sixty (60) hamsters were randomized into six groups of ten animals each, prior to irradiation. Each group was assigned a different treatment as listed in Table 18. Animals were dosed with 60 mg/kg 5-FU on days −4 and −2 and were acutely irradiated on the left buccal mucosa on day 0. Animals were treated once daily with CG53135-05 E. coli purified product IP 6, 12, 24 or 48 mg/kg/day, on day 1 only or 12 mg/kg/day on days 1 and 2, following acute radiation. Mucositis was evaluated on alternate days beginning on day 6 and continued until the conclusion of the experiment on day 28 (i.e., days 8, 10, 12, 14, 16, 18, 20, 22, 24, 26 & 28).

TABLE 18
Treatment Groups

| Group | Number of | | Treatment Volume |
|---|---|---|---|
| Number | Animals | Treatment | Schedule* |
| 1 | 10 males | Untreated Control | Day 1 |
| 2 | 10 males | 6 mg/kg CG53135-05 E. coli | Day 1 |
| | | purified product, ip | |
| 3 | 10 males | 12 mg/kg CG53135-05 E. coli | Day 1 |
| | | purified product ip | |
| 4 | 10 males | 24 mg/kg CG53135-05 E. coli | Day 1 |
| | | purified product, ip | |
| 5 | 10 males | 48 mg/kg CG53135-05 E. coli | Day 1 |
| | | purified product ip | |
| 6 | 10 males | 12 mg/kg CG53135-05 E. coli | Day 1 & 2 |
| | | purified product, ip | |

*Mucositis was evaluated on alternate days beginning on day 6 and every second day until the conclusion of the experiment on day 28 (i.e., days 8, 10, 12, 14, 16, 18, 20, 22, 24, 26, & 28).

Chemotherapy/Radiation Model of Oral Mucositis: the 5-FU/acute irradiation model for oral mucositis in hamsters is an experimental model designed to extend the clinical observations made with the acute radiation model for mucositis (Oral Surg Oral Med Oral Pathol 69(4):437 (1990)). The earlier acute radiation model has proven to be an accurate, efficient and cost-effective technique to provide a preliminary evaluation of anti-mucositis compounds including growth factors and cytokines (see e.g., Oral Oncol 36(4):373-381 (2000), Cytokine 9(8):605-612 (1997); Oral Oncol 33(1):47-54 (1997)).

Mucositis was induced using 5-fluorouracil, delivered as intraperitoneal (IP) doses (60 mg/kg) on days −4 and −2. A single dose of radiation (30Gy/dose) was administered to all animals on day 0. Radiation was generated with a 160 kilovolt potential (18.75-ma) source at a focal distance of 21 cm, hardened with a 3.0 mm Al filtration system. Irradiation targeted the left buccal pouch mucosa at a rate of 1.32Gy/minute. Prior to irradiation, animals were anesthetized with an IP injection of ketamine (160 mg/kg) and xylazine (8 mg/kg). The left buccal pouch was everted, fixed and isolated using a lead shield. This resulted in ulcerative oral mucositis that peaked around day 14.

Evaluation of Mucositis: for the evaluation of mucositis, the animals were anesthetized with an inhalation anesthetic, and the left pouch was everted. Mucositis was scored visually by comparison to a validated photographic scale, ranging from 0 for normal, to 5 for severe ulceration. The scale is described in Table 19.

TABLE 19
Description of Mucositis Score Values

| Score | Description: |
|---|---|
| 0 | Pouch completely healthy. No erythema or vasodilation. |
| 1 | Light to severe erythema and vasodilation. No erosion of mucosa. |
| 2 | Severe erythema and vasodilation. Erosion of superficial aspects |
| | of mucosa leaving denuded areas. Decreased stippling of mucosa. |
| 3 | Formation of off-white ulcers in one or more places. Ulcers may |
| | have a yellow/gray due to pseudomembrane. Cumulative size |
| | of ulcers should equal about ¼ of the pouch. Severe |
| | erythema and vasodilation. |
| 4 | Cumulative size of ulcers should equal about ½ of the pouch. Loss |
| | of pliability. Severe erythema and vasodilation. |
| 5 | Virtually all of pouch is ulcerated. Loss of pliability (pouch can |
| | only partially be extracted from mouth) |

A score of 1-2 is considered to represent a mild stage of the disease, whereas a score of 3-5 is considered to indicate moderate to severe mucositis. Following clinical scoring, a photograph was taken of each animal's mucosa using a standardized technique. At the conclusion of the experiment, all film was developed and the photographs randomly numbered for blinded scoring. Two independent, trained evaluators graded the photographs in blinded fashion using the above-described scale. For each photograph, the final blinded score was the average of the score assigned by the two independent evaluators. The scores from the blinded photographic evaluation were statistically analyzed.

Weights and Survival: each hamster was weighed daily for the period of the study (i.e., day −4 to day 28). Weight and survival was monitored and recorded in order to assess possible differences amongst treatment groups as an indication for mucositis severity and/or possible toxicity resulting from the treatments. If appropriate, survival was analyzed using a Kaplan Meier log-rank analysis. Differences in weight gain were assessed using a One-Way ANOVA analysis of the area under the curve (AUC) values for the percentage weight gain for individual animals, with a critical value of 0.05.

Evaluation of Activity: the effect of each treatment on mucositis compared to the control group was assessed using a Chi-squared (×2) analysis of the number of animal days with a score of three or higher, and by using the Mann-Whitney Rank Sum test to compare the blinded mucositis scores for each group on each day the evaluations were performed. In each case, treatment groups were compared to the control group, with a critical value of 0.05. For the Mann-Whitney Rank Sum test, two days of statistically significant improvement are generally regarded as the minimum improvement necessary for a positive result.

Results

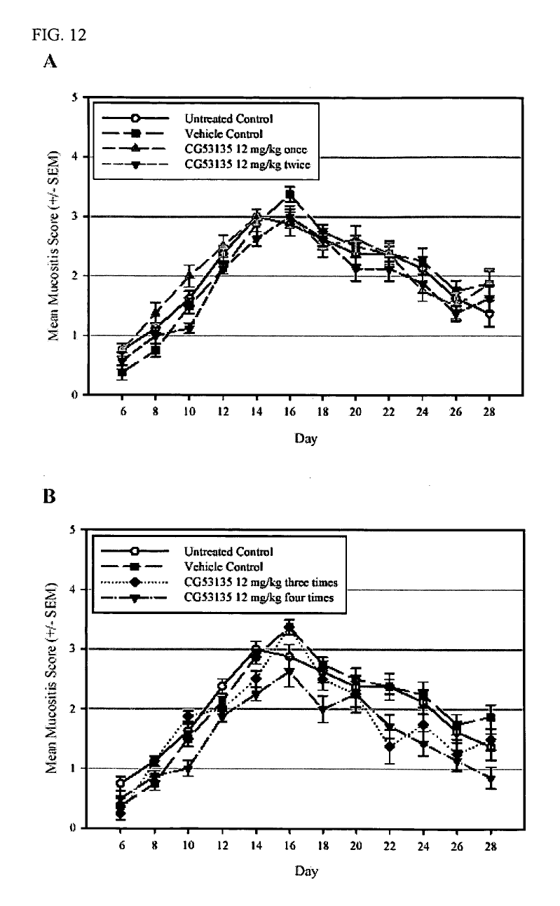

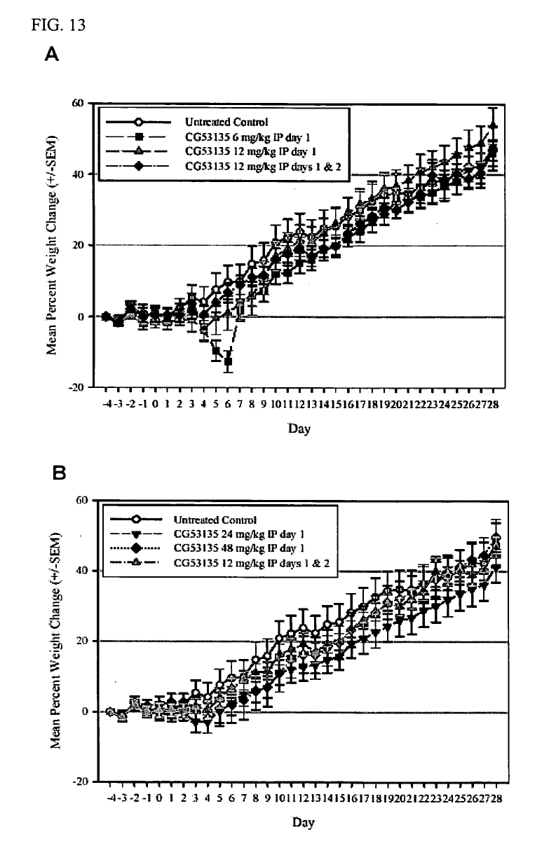

Mucositis: the mean daily mucositis scores were calculated for each group and are shown in FIG. 11. The peak of mucositis in the control group was on day 14 when the mean score for this group reached 3.2. All of the groups treated with CG53135-05 E. coli purified product had their peak scores on day 16, which ranged from a high of 3.0 in the groups treated with CG53135-05 E. coli purified product at 24 mg/kg or 48 mg/kg on day 1 to a low of 2.63 in the group treated with CG53135-05 E. coli purified product at 12 mg/kg on day 1. To evaluate the mucositis scores, an analysis of the number of days with a score of 3 or higher was performed, using the Chi-squared test. The results of this analysis are shown in Table 20 and FIG. 11. Further, FIG. 12 depicts the duration of severe mucositis in animals with a mucositis score of >3 as calculated by the chi-square analysis. Both groups treated with CG53135-05 E. coli purified product at 12 mg/kg showed a significant reduction in the number of days with a score of 3 or higher, with the group treated on day 1 only having slightly more significance (P=0.003) than the group treated on days 1 and 2 (P=0.018). The group treated with CG53135-05 E. coli purified product at 6 mg/kg on day 1 showed some improvement, but failed to reach significance (P=0.092). The groups treated with CG53135-05 E. coli purified product at 24 mg/kg and 48 mg/kg were essentially the same as controls in this test.

TABLE 20
Chi Squared analysis of number of days animals had a mucositis score of 3 or higher

| | | | | % | Chi Sq. | P |
|---|---|---|---|---|---|---|
| Group | Days >= 3 | Days < 3 | Total Days | Days >= 3 | vs control | Value |
| Untreated control | 94 | 140 | 234 | 40.2 | — | — |
| CG53135-05 E. coli | 70 | 148 | 218 | 32.1 | 2.8330 | 0.092 |
| purified product 6 mg/kg | | | | | | |
| IP Day 1 | | | | | | |
| CG53135-05 E. coli | 52 | 148 | 200 | 26.0 | 9.0760 | 0.003 |
| purified product 12 mg/kg | | | | | | |
| IP Day 1 | | | | | | |
| CG53135-05 E. coli | 80 | 136 | 216 | 37.0 | 0.3420 | 0.558 |
| purified product 24 mg/kg | | | | | | |
| IP Day 1 | | | | | | |
| CG53135-05 E. coli | 94 | 126 | 220 | 42.7 | 0.2090 | 0.647 |
| purified product 48 mg/kg | | | | | | |
| IP Day 1 | | | | | | |
| CG53135-05 E. coli | 70 | 168 | 238 | 29.4 | 5.5590 | 0.018 |
| purified product 12 mg/kg | | | | | | |
| IP Day 1 and 2 | | | | | | |

To examine the levels of clinically significant mucositis, as defined by presentation with open ulcers (score >3), the total number of days in which an animal exhibited an elevated score was summed and expressed as a percentage of the total number of days scored for each group. Statistical significance of observed differences was calculated using chi-square analysis. Significant improvement is highlighted in the table.

Further analysis of the significance of the differences in the mucositis scores was performed by using the Mann-Whitney Rank-Sum test to compare the test groups with the control group on each day of evaluation. The results of this analysis are shown in Table 21 which indicates that the group treated with CG53135-05 E. coli purified product at 6 mg/kg on day 1 only showed significant improvement relative to controls on days 14 (P=0.010) and 26 (P=0.031). The group treated with CG53135-05 E. coli purified product at 12 mg/kg on day 1 only showed significant improvement relative to controls on days 14 (P=0.011), 16 (P=0.031), 18 (P=0.005), and 20 (P=0.037). The group treated with CG53135-05 E. coli purified product at 24 mg/kg did not show any significant improvement relative to controls. The group treated with CG53135-05 E. coli purified product at 48 mg/kg showed significant improvement on day 12 (P=0.035) but also showed significant worsening on days 26 (P=0.036) and 28 (P=0.006). The group treated with CG53135-05 E. coli purified product at 12 mg/kg/day on days 1 and 2 showed significant improvements relative to controls on days 14 (P=0.010) and 18 (P=0.045). Since the standard for meaningful improvement in this test is 2 days of statistically significant improvement in the mucositis score relative to controls, the groups treated on day 1 with CG53135-05 E. coli purified product at either 6 mg/kg or 12 mg/kg, and the group treated with CG53135-05 E. coli purified product at 12 mg/kg/day on days 1 and 2 showed meaningful improvements.

TABLE 21
Mucositis scores as performed by using the Mann-Whitney Rank-Sum test

| Group | Day | | | | | | | | | | | |
|---|---|---|---|---|---|---|---|---|---|---|---|---|
| Comparison | 6 | 8 | 10 | 12 | 14 | 16 | 18 | 20 | 22 | 24 | 26 | 28 |
| CG53135-05 E. coli | 0.597 | 0.735 | 0.826 | 0.298 | 0.010 | 0.606 | 0.324 | 0.164 | 0.224 | 0.736 | 0.031 | 0.202 |
| purified | | | | | | | | | | | | |
| product | | | | | | | | | | | | |
| 6 mg/kg day 1 | | | | | | | | | | | | |
| vs control | | | | | | | | | | | | |
| CG53135-05 E. coli | 0.989 | 0.595 | 0.042 | 0.164 | 0.011 | 0.031 | 0.005 | 0.037 | 0.232 | 0.762 | 0.576 | 0.347 |
| purified | | | | | | | | | | | | |
| product | | | | | | | | | | | | |
| 12 mg/kg day 1 | | | | | | | | | | | | |
| vs control | | | | | | | | | | | | |
| CG53135-05 E. coli | 0.781 | 0.129 | 0.736 | 0.104 | 0.104 | 0.553 | 0.115 | 0.988 | 0.298 | 0.356 | 0.297 | 0.388 |
| purified | | | | | | | | | | | | |
| product | | | | | | | | | | | | |
| 24 mg/kg day 1 | | | | | | | | | | | | |
| vs control | | | | | | | | | | | | |
| CG53135-05 E. coli | 0.797 | 0.989 | 0.393 | 0.035 | 0.101 | 0.553 | 0.295 | 0.224 | 0.780 | 0.164 | 0.036 | 0.006 |
| purified | | | | | | | | | | | | |
| product | | | | | | | | | | | | |
| 48 mg/kg day 1 | | | | | | | | | | | | |
| vs control | | | | | | | | | | | | |
| CG53135-05 E. coli | 0.284 | 0.161 | 0.284 | 0.106 | 0.010 | 0.144 | 0.045 | 0.260 | 0.163 | 0.424 | 0.989 | 0.456 |
| purified | | | | | | | | | | | | |
| product | | | | | | | | | | | | |
| 12 mg/kg day 1 | | | | | | | | | | | | |
| and 2 vs control | | | | | | | | | | | | |

Significance of Group Differences Observed in Daily Mucositis Scores (Rank Sum Test). This nonparametric statistic is appropriate for the visual mucositis scoring scale. The p values for each calculation are shown. Significant improvement is highlighted.

Survival: five animal deaths occurred during the study. The deaths occurred on day 4 in the group receiving CG53135-05 E. coli purified product at 24 mg/kg, day 7 in the 6 mg/kg group, days 9 and 11 in the 12 mg/kg on day 1 only group and day 11 in the 48 mg/kg group. No deaths were observed in either the control group or in the group receiving CG53135-05 E. coli purified product at 12 mg/kg/day on days 1 and 2. Survival was consistent with the death rate usually observed in the chemotherapy/radiation model.

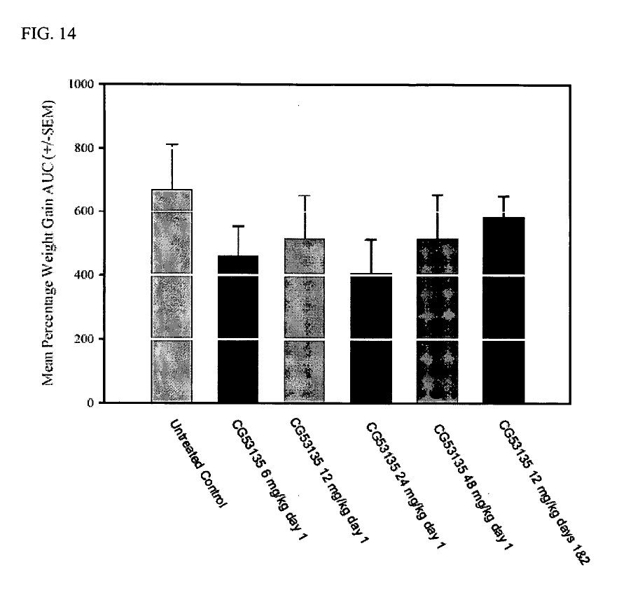

Weight Change: the mean daily percentage weight change for each group is shown in FIG. 13. The overall increase in weight during the course of this study for animals in the untreated control group was 47.5%, compared with 45.9% in the group treated with CG53135-05 at 6 mg/kg in day 1, 53.8% in the group treated with CG53135-05 E. coli purified product at 12 mg/kg in day 1, 41.2% in the group treated with CG53135-05 E. coli purified product at 24 mg/kg in day 1, 49.7% in the group treated with CG53135-05 E. coli purified product at 48 mg/kg in day 1, and 46.9% in the group treated with CG53135-05 E. coli purified product at 12 mg/kg on days 1 and 2. Analysis of group weight gain was done by calculation of the area under the curve (AUC) for each animal. One-Way ANOVA analysis of group AUC values for weight among all study groups indicated that there were no significant differences between any groups in the study (P=0.687). A mean comparison of AUC values for each group in this study is shown in FIG. 14. This result indicates that the animals in groups treated with CG53135-05 E. coli purified product gained weight in a manner that was equivalent to those in the untreated control group.

### 6.11 Example 11

### Effect of CG53135 on Treatment of Established Oral Mucositis in Hamster Chemo/Radiation Model (N-318)

Animal, type and age, and the chemotherapy/radiation model are same as described in Section 6.10. Protein concentrations in this example were measured by Bradford assay.

Sixty (60) hamsters were randomized into six (6) groups of ten (10) animals each, prior to irradiation. Each group was assigned a different treatment as listed and treated with CG53135-05 E. coil purified product,12 mg/kg IP as indicated in Table 22. In this study, animals were dosed with 60 mg/kg 5-FU on days −4 and −2, followed by an acute radiation dose of approximately 30Gy on Day 0 in order to produce severe mucositis around Day 15. The duration of this study was 35 days. The treatment schedule and dosing started after animals reach an oral mucositis score of 2. In addition to the mucositis scoring, this study evaluated the occurrence of diarrhea, weight loss and death for each animal in the experimental groups.

TABLE 22
Treatment Groups

| Group | Number of | | Treatment |
|---|---|---|---|
| Number | Animals | Treatment | Schedule |
| 1 | 8 males | Untreated Control | None |
| 2 | 8 males | Vehicle control | Once Daily (3x) |
| | | | on OM score of 2 |
| 3 | 8 males | 12 mg/kg CG53135-05 E. coli | Once Daily (1x) |
| | | purified product, ip, once | on OM score of 2 |
| 4 | 8 males | 12 mg/kg CG53135-05 E. coli | Once Daily (2x) |
| | | purified product, ip, twice | on OM score of 2 |
| 5 | 8 males | 12 mg/kg CG53135-05 E. coli | Once Daily (3x) |
| | | purified product, ip, thrice | on OM score of 2 |
| 6 | 8 males | 12 mg/kg CG53135-05 E. coli | Once Daily (4x) |
| | | purified product, ip, four times | on OM score of 2 |

Mucositis was evaluated on alternate days beginning on day 6 and every second day until the conclusion of the experiment on day 28 (i.e., days 8, 10, 12, 14, 16, 18, 20, 22, 24, 26, & 28).

Mucositis was induced in hamsters. The end points, mucositis, weights and survival, were evaluated. Statistics applied were Chi-squared analysis and Mann-Whitney Rank Sum test. All the three parameters are described in Section 6.10.

Results

Mucositis: in the untreated control group, the peak of mucositis occurred on day 14 with a mean score of 3. In the vehicle control group the peak of mucositis occurred on day 16 with a mean score of 3.4. The groups receiving CG53135-05 E. coli purified product 12 mg/kg IP on the first and second days after reaching a score of 2 showed similar patterns of mucositis scores to the control groups (FIG. 12A). The groups that received CG53135-05 E. coli purified product 12 mg/kg IP on the third and fourth days after reaching a score of 2 showed a reduction in mucositis scores relative to the control groups, predominantly after the peak of mucositis (FIG. 12B).

The differences in mucositis scores between the groups were evaluated by comparing the number of days with a score of 3 or higher using a Chi-Squared test. In the untreated control group, 32.3% of the animals days evaluated had a score of 3 or higher, compared with 41.1% of animals days in the vehicle control group. As a result of this difference between the two control groups, two treated groups (groups receiving CG53135-05 E. coli purified product on 2 and 3 days after reaching a score of 2) showed a significant improvement when compared with the vehicle control, but not when compared to the untreated controls. For the group treated with CG53135-05 E. coli purified product for 2 days, the P values were 0.347 compared to untreated controls and 0.007 when compared to the vehicle controls, and for the group treated with CG53135-05 E. coli purified product for 3 days the P values were 0.580 compared to untreated controls and 0.020 when compared to vehicle controls. The group treated with CG53135-05 E. coli purified product for four days after reaching a score of 2 showed significant improvement when compared with both the untreated (P=0.003) and vehicle (P<0.001) controls. The group treated with CG53135-05 E. coli purified product on only one day after reaching a score of 2 did not show significance when compared with either control group.

Further evaluation of the significance of the differences seen between the control and treated groups was performed by using a Mann-Whitney rank sum test to evaluate the mucositis scores for each group on each day that scores were obtained. In this analysis, the different treatment groups were compared with either the untreated control group or the vehicle control group. The results of this comparison with the untreated control group showed that there was a statistically significant difference between the group treated for 2 days and the untreated control group on day 10 only (P=0.011). There were statistically significant differences between the group treated for 3 days and the untreated control group on days 14 (P=0.036) and 22 (P=0.013). Statistically significant differences between the group treated for 4 days and the untreated control were seen on days 10 (P=0.009), 12 (P=0.029), 14 (P=0.002) 22 (P=0.021) and 24 (P=0.032). No statistically significant differences were seen between the group treated with CG53135 on a single day and the untreated control group.

The results of the rank sum comparison between the vehicle control group showed that there was a statistically significant difference between the group treated on 3 days and the vehicle control group on days 14 (P=0.020) and 22 (P=0.020). Statistically significant differences between the group treated on 4 days and the vehicle control were seen on days 10 (P=0.036), 14 (P<0.001), 18 (P=0.024), 22 (P=0.048), 24 (P=0.021), 26 (P=0.048) and 28 (P=0.004). No statistically significant differences were seen between the groups treated with CG53135 on a single day, or 2 days and the vehicle control group.

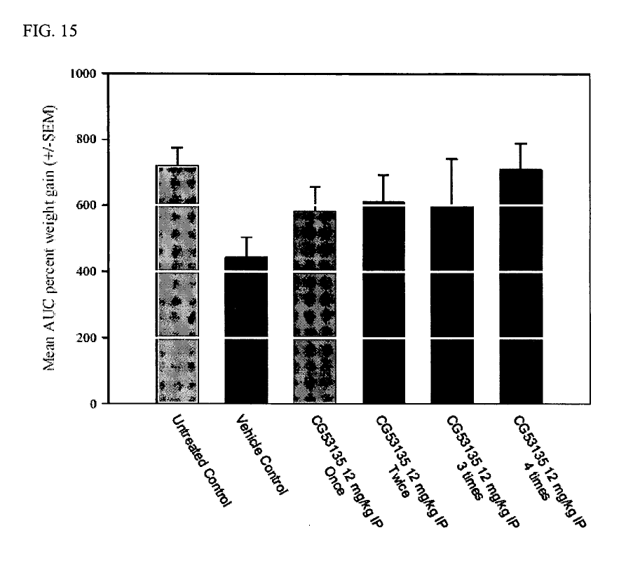

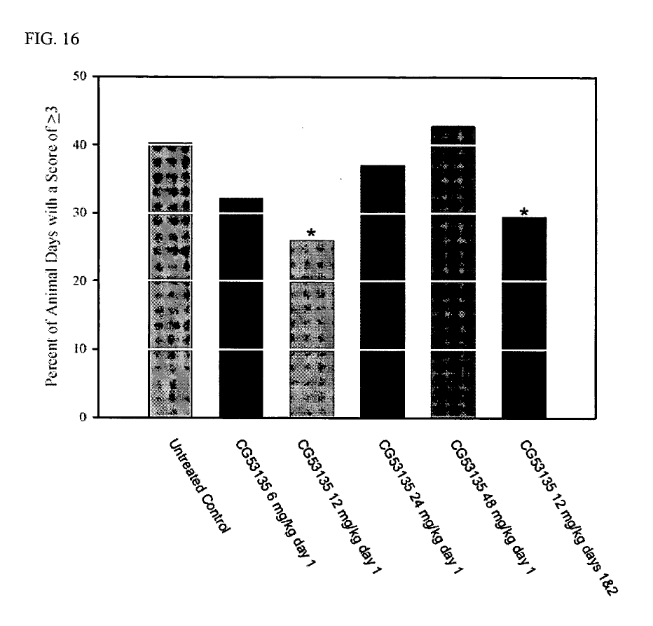

Weight Change: animals in the untreated control group gained an average of 50.5% of their starting body weight by the end of the study. The vehicle control group had the lowest mean gain in weight during the study, gaining an average of 41.1%. The group that received four daily doses of CG53135-05 E. coli purified product had the largest gain in weight during the study at 53.4%, while the group that received one, two and three daily doses gained an average of 48.1%, 46.8% and 44.4% respectively. These differences were evaluated by calculating the area under the curve (AUC) for the percent daily weight gain for each animal and then evaluating the AUC values using a one way ANOVA test. No significant differences were seen between the groups (P=0.266). The mean AUC data is shown in FIG. 15.

### 6.12 Example 12

### CG53135 Can be Used Safely as a Single Dose Therapy for Mucositis in Human Patients (Studies C-214 and C-325)

Safety, tolerability, and pharmacokinetic assessment of a CG53135-05 E. coli purified product (in the formulation as described in Section 6.17.1, referred as “CG53135-05 drug substance” in this example) administered intravenously in human patients with advanced (Stage 4) cancer (single rising-dose tolerance) was conducted. The goal of this dose-escalating tolerance study was to assess the safety, tolerability and pharmacokinetics of CG53135-05 drug substance in cohorts of four patients at 0.03, 0.1, 0.33, and 1 mg/kg (UV). Dose escalation was stopped due to tolerability information at 0.33 mg/kg delivered in 15 minutes (Phase I study C-325) and the protocol was amended to add a 0.2 mg/kg dose.

During the trial, oral mucosa was examined by experienced study staff and assigned a mucositis score by both the World Health Organization (WHO) OM scoring system and the Oral Mucositis Assessment Scale (OMAS). See, WHO Handbook 1979 WHO. WHO Handbook for Reporting Results for Cancer Treatment. In: WHO Offset Publication No. 48. Geneva, Switzerland: World Health Organization; 1979. The OMAS provides a more quantitative assessment of injury. After discharge, patients were provided with diaries where they noted a single WHO score for each day. Study staff instructed patients on how to self-assess and assign a score for oral mucositis.

TABLE 23
WHO Scoring System:

| Grade 0 | Grade 1 | Grade 2 | Grade 3 | Grade 4 |
|---|---|---|---|---|
| Normal | Erythema and | Ulceration, but | Ulceration, diet | Ulceration of |
| | soreness | can eat solid | limited to | such severity |
| | | foods | liquids | that patient |
| | | | | requires |
| | | | | parenteral |
| | | | | feeding |

A value of the OMAS system is obtained by summing the erythema and ulceration/pseudomembrane scores.

TABLE 24
OMAS Scoring System

| | 0 | 1 | 2 | 3 |
|---|---|---|---|---|
| Erythema | None | Mild/moderate | Severe | |
| | | erythema | erythema | |
| Ulceration/ | No | Cumulative | Cumulative | Cumulative |
| psuedomembrane | lesions | surface area of | surface area | surface |
| | | lesion < 1 cm² | of lesion > | area of |
| | | | 1 cm² and < | lesion is > |
| | | | 3 cm² | 3 cm² |

Eleven patients have received the CG53135-05 drug substance at 0.03 mg/kg (n=4), 0.1 mg/kg (n=6), and 0.2 mg/kg (UV) (n=1) as a single 100-ml intravenous infusion administered 3 days after completion of the CT. Tolerability information is available for all 11 patients. Full clinical data from nine patients are available.

Preliminary pharmacokinetic data demonstrated plasma exposure with an average Cmax of 564.3 ng/ml at the 0.03 mg/kg (UV) dose level (n=3; range 175.6-1192.6 ng/ml) and 564.7 ng/ml at the 0.1 mg/kg (UV) dose level (n=3; range 420.9-797.5 ng/ml). After infusion, the CG53135-05 drug substance reached maximum plasma concentration within 1hour (15 to 35 minutes after completion of infusion). The mean terminal exponential half-life was 49 minutes (range: 16.2-87 minutes, n=5). No patients discontinued the trial due to adverse experiences. Adverse events (number of patients) that may be related to the study drug include: nausea (2); chills (2); fever (2); vomiting (1); dizziness (1); photopsia (1) (vision-“lights flashing” on day 15) and astigmatism (1) (mild astigmatism on day 28); neuropathy (1) (on soles of the feet on day 15); tachycardia (1); headache (1); and asymptomatic, single premature atrial complex noted on ECG (1). All reported incidences were mild to moderate. No Grade 3 or 4 laboratory toxicity associated with the study drug was noted. Among the 11 patients who have received the drug through Sep. 3, 2004, six serious adverse events determined to be unrelated to study drug were noted from 3 patients. These events included cancer progression (n=2), catheter infection, small intestinal obstruction, esophagitis/mucositis and neutropenic fever.

Among the 11 patients that completed the study, six patients did not develop oral mucositis. Four patients developed Grade 1 (n=1) or Grade 2 (n=3) oral mucositis. One patient with a Grade 3 oral mucositis was observed. No patients required total parenteral nutrition. Patients receiving CPT-11 typically have a high incidence of diarrhea. In this trial, 7 of the patients received CPT-11 as part of CT and only two patients (both received 0.03 mg/kg (UV) of CG53135-05 drug substance) experienced mild to moderate diarrhea and only 1 patient developed diarrhea immediately after receiving CG53135-05 drug substance treatment. We concluded that the CG53135-05 drug substance was well-tolerated with single dose administration at 0.03, 0.1,and 0.2 mg/kg (UV).

A concurrent single rising-dose, phase I trial (study C-325) in autologous stem cell transplant patients is ongoing and 27 patients have been treated with CG53135-05 drug substance. 22 patients (pts) (ages 25-75) undergoing HDCT with PBSCT have completed the study with escalating doses of CG53135-05 drug substance, including (number of patients): 0.03 mg/kg (2), 0.1 mg/kg (10), 0.2 mg/kg (8), and 0.33 mg/kg (2). Patients were treated for: multiple myeloma (n=11), non-Hodgkin's lymphoma (n=9), acute myelogenous leukemia (n=1), and desmoplastic round cell tumor (n=1) and were treated with conditioning regimens including melphalan (Mel 200), cyclophosphamide, carmustine and etoposide (CBV), carboplatin and thiotepa (CT), and busulfan/cyclophosphamide (targeted BuCy). The primary objective of the trial was to evaluate safety, tolerability and pharmacokinetics of the CG53135-05 drug substance. Patients were also scored daily for presence of OM using both the WHO and OMAS grading scales (Tables 23 and 24, supra). Among the 22 patients that completed the study, 8 patients experienced no OM (including 4 Mel 200 pts); 10 patients experienced only WHO grade 1 (n=7) or grade 2 (n=3) OM, while 4 patients experienced severe OM of Grade 3 (n=3) or Grade 4 (n=1). 1 patient experiencing grade 4 OM required TPN for 4 days. Patients tolerated the study drug well with no significant side effects up to a dose of 0.33 mg/kg. At that dose, 2 patients experienced an infusional reaction consisting of fevers, nausea, and mild hypotension. Preliminary Pharmacokinetic results from 13 patients confirmed dose dependent plasma exposure with an average Cmax of 135.5 ng/ml, 343.3ng/ml, and 658.3ng/ml at dose levels of 0.1, 0.2, and 0.33 mg/kg, respectively. The median day of neutrophil engraftment (as determined by ANC>500/uL) occurred on day 13 after stem cell infusion. Preliminary data suggest that CG53135-05 drug substance is well tolerated in PBSCT patients at doses up to 0.33 mg/kg with apparent clinical effects in ameliorating or preventing OM. 18/22 patients, thus, avoided (WHO Grades 3-4) mucositis following HDCT. A larger Phase II clinical trial will be initiated to evaluate the efficacy of CG53135-05 drug substance in preventing HDCT-induced OM.

In conclusion, the CG53135-05 drug substance was generally well tolerated among the 38 patients that were administered in the two phase I trials to date. The doses tested were 0.03, 0.1, 0.2 and 0.3 mg/kg (UV). Infusional reactions when the 0.33 mg/kg of drug was administered over 15 minutes coupled with apparent activity observed at lower doses led to a discontinuation of this dose level. No other consistent drug-related or apparent dose-related adverse events or laboratory abnormals have been observed. No study drug-related serious adverse events were observed. Sufficient information on tolerability and preliminary activity is considered to be present to utilize the 0.03, 0.1 and 0.2 mg/kg (UV) doses in Phase II testing.

### 6.13 Example 13

### Multiple Doses of CG53135 for Preventing or Treating Mucositis

Preclinical studies using a validated hamster oral mucositis (“OM”) model have supported the exploration of the effects of CG53135 in treating patients with active OM. The results of the study further demonstrated that multiple-dosing regimen was more efficacious in reducing the duration and severity of OM (Study N-318 in Example 6.11). A third phase I clinical trial can be conducted to assess the safety of 3 consecutive daily doses of CG53135-05 drug substance at 0.03, 0.1, or 0.2 mg/kg (UV) on patients with active OM from CT and/or TBI as conditioning for AHSCT. In addition, OM scores, pharmacokinetics, and pharmacodynamics (PD) will be assessed.

The proposed single doses of 0.03, 0.1, or 0.2 mg/kg (UV) have been administered on cancer patients in the two phase I clinical trials conducted to assess safety and tolerability of single dose CG53135-05 drug substance. CG53135-05 drug substance was well tolerated with the proposed trial doses among the patients that have completed the trial. The third phase I trial proposes daily administration of pharmacologic doses of CG53135-05 drug substance for 3 consecutive days. Given the limited duration of therapy of three days and the potential benefit of the information derived from the study (positive safety data will justify further detailed exploration of these safety issues), the risk-benefit profile is considered to be favorable.

Blood samples will be collected to monitor changes in hematology and clinical chemistry and to measure levels of CG53135-05 drug substance and antibody to CG53135. Urine will be collected for urinalysis parameters. Electrocardiogram (ECG) as well as physical and eye examinations will also be performed for assessment of safety. Blood samples will be collected to assess biomarkers of drug activity. In the context of this study, biomarkers are defined as characteristics that are objectively measured and evaluated as indicators of pharmacologic responses to treatment with CG53135-05 drug substance. Biomarkers that may be monitored include cytokines.

### 6.14 Example 14

### Prevention of Oral Mucositis Derived From Chemotherapy or Combination of Chemotherapy and Radiation Therapy

A phase II clinical trial can be conducted to evaluate the efficacy of a CG53135-05 drug substance in preventing oral mucositis from cancer patients that receive chemotherapy or radiation therapy. This will be a double blind placebo controlled study with a single dose of a CG53135-05 drug substance at 0.03, 0.1, or 0.2 mg/kg (UV) intravenously administered. The World Health Organization (WHO) OM scoring system will be used to measure efficacy.

Patients receiving an autologous stem cell transplant following myelo-ablative chemotherapy and/or Total Body Irradiation (TBI) will be screened to enroll patients. Patients will receive autologous stem cells twenty-four hours after completion of the chemotherapy and/or total body irradiation (TBI) regimen.

Patients will receive a CG53135-05 drug substance at least twenty-four hours after completion of the stem cell infusion. Patients will be monitored post-drug administration until discharged. Patients will return for follow-up 2 weeks after discharge, 30 days and 90 days following the infusion of the CG53135-05 drug substance.

Blood samples will be collected to measure levels of the CG53135, antibody to CG53135, and to monitor changes in hematology and clinical chemistry. Urine will be collected for urinalysis parameters. Electrocardiogram (ECG) and physical examinations will also be performed for assessment of safety. Blood samples will be collected to assess biomarkers of drug activity. The primary end point of efficacy will be demonstrated as the duration and severity of oral mucositis. The secondary end points will include days with fever, total parenteral nutrition, number of infections, use of intravenous narcotic analgesics, time to engraftment, time to discharge, incidence and duration of diarrhea, as well as mortality/progression-free disease at 90 days.

### 6.15 Example 15

### Treatment of Oral Mucositis Derived From Chemotherapy or Combination of Chemotherapy and Radiation Therapy

A phase II clinical trial may be conducted to evaluate the efficacy of a CG53135-05 drug substance in treating oral mucositis from cancer patients that receive chemotherapy or radiation therapy. This will be a double blind placebo controlled study with a single dose or multiple doses of a CG53135-05 drug substance intravenously administered. The World Health Organization (WHO) OM scoring system will be used to measure efficacy of the treatment.

Patients will be monitored daily for the onset of oral mucositis. Treatment with a CG53135-05 drug substance will be initiated when patients develop oral mucositis with a WHO score of 1 or 2.

A CG53135-05 drug substance will be administered as a single dose or multiple doses. Patients will be monitored post-drug administration until discharged. Patients will return for follow-up 2 weeks after discharge, 30 days and 90 days following the infusion of the drug.

Blood samples will be collected to measure levels of CG53135, antibody to CG53135, and to monitor changes in hematology and clinical chemistry. Urine will be collected for urinalysis parameters. Electrocardiogram (ECG) and physical examinations will also be performed for assessment of safety. Blood samples will be collected to assess biomarkers of drug activity. The primary end point of efficacy will be demonstrated as the duration and severity of oral mucositis. The secondary end points will include days with fever, total parenteral nutrition, number of infections, use of intravenous narcotic analgesics, time to engraftment, time to discharge, incidence and duration of diarrhea, as well as mortality/progression-free disease at 90 days.

### 6.16 Example 16

### CG53135 Reduces the Incidence, Length and Severity of Radiation-Induced Diarrhea (N-438)

This study was performed to evaluate the activity of CG53135 against gastrointestinal injury induced by whole body irradiation as measured by diarrhea incidence and gut morphology. Protein concentrations in this example were measured by UV absorbance.

Materials and Methods:

Dosing: Mice were weighed and then dosed with CG53135-05 E. coli purified product (4 or 16 mg/kg) or untreated. Dosing occurred as described in Tables 1 & 2. Each group of 20 animals was irradiated as per table below. All dosing of CG53135-05 E. coli purified product on day 0 was immediately after irradiation. No anesthesia was administered.

Intestinal Crypt Cell Damage Induction: Mice underwent whole body irradiation at a dose of 14 or 14.5 Gy delivered at a dose rate of 0.7Gy/min. Animals were followed for diarrhea incidence throughout the study period. After 6 days, animals were sacrificed, and the intestinal tract of the mice was harvested for histological analysis.

Body Weight: Every day for the period of the study, each animal was weighed and its survival recorded, in order to assess possible differences in animal weight among treatment groups as an indication of response to exposure to ionizing radiation.

Animals Found Dead or Moribund: Animals were assessed 2×/day from Day 3 onwards in order to accurately assess diarrhea onset/progression and detect moribund animals prior to death. Such moribund animals were sacrificed by cervical dislocation. The ileum and mid-colon were removed and fixed in formalin, embedded in paraffin (1 animal per block, two tissues per block) for storage and future analysis/IHC if required. No tissue was removed from animals found dead.

TABLE 25
Study Design

| Group | Number of | | | Treatment | Volume |
|---|---|---|---|---|---|
| Number | Animals | Induction | Treatment | Schedule* | (mL) |
| 1 | 20 males | 14 Gy | None | None | Adjust per body |
| | | Day 0 | | | weight |
| 2 | 20 males | 14 Gy | CG53135-05 E. coli | Day −1, 0, 1 | Adjust per body |
| | | Day 0 | purified product, | | weight |
| | | | 4 mg/kg, IP | | |
| | | | (qd × 3) | | |
| 3 | 20 males | 14 Gy | CG53135-05 E. coli | Day −1, 0, 1 | Adjust per body |
| | | Day 0 | purified product, | | weight |
| | | | 16 mg/kg, IP | | |
| | | | (qd × 3) | | |
| 4 | 20 males | 14 Gy | CG53135-05 E. coli | Day 1 | Adjust per body |
| | | Day 0 | purified product, | | weight |
| | | | 4 mg/kg, IP | | |
| | | | (q6h × 4) | | |
| 5 | 20 males | 14.5 Gy | None | None | Adjust per body |
| | | Day 0 | | | weight |
| 6 | 20 males | 14.5 Gy | CG53135-05 E. coli | Day −1, 0, 1 | Adjust per body |
| | | Day 0 | purified product, | | weight |
| | | | 4 mg/kg, IP | | |
| | | | (qd × 3) | | |
| 7 | 20 males | 14.5 Gy | CG53135-05 E. coli | Day −1, 0, 1 | Adjust per body |
| | | Day 0 | purified product, | | weight |
| | | | 16 mg/kg, IP | | |
| | | | (qd × 3) | | |

TABLE 26
Test Article Requirements

| | | | | | | | Conc. of | Desired | Volume | Volume of |
|---|---|---|---|---|---|---|---|---|---|---|
| | | Dose | | | | | stock | Conc of | of dosing | stock |
| | | (mg/kg) | | | Mass of | | solution | Dosing | solution | solution |
| Type of | | from | # of | # of | Animal | Admin | by A₂₈₀ | solution | required | required for |
| Solution | Group # | Conc A₂₈₀ | Animals | doses | (kg) | vol (mL/kg) | (mg/mL) | (mg/mL) | (mL) | dilution (mL) |
| CG53135 | 1 | 0 | 20 | 0 | 0.025 | 10 | 10.2 | 0.000 | 0.000 | 0.000 |
| CG53135 | 2 | 4 | 20 | 3 | 0.025 | 10 | 10.2 | 0.400 | 18.750 | 0.735 |
| CG53135 | 3 | 16 | 20 | 3 | 0.025 | 10 | 10.2 | 1.600 | 18.750 | 2.941 |
| CG53135 | 4 | 4 | 20 | 4 | 0.025 | 10 | 10.2 | 0.400 | 25.000 | 0.980 |
| CG53135 | 5 | 0 | 20 | 0 | 0.025 | 10 | 10.2 | 0.000 | 0.000 | 0.000 |
| CG53135 | 6 | 4 | 20 | 3 | 0.025 | 10 | 10.2 | 0.400 | 18.750 | 0.735 |
| CG53135 | 7 | 16 | 20 | 3 | 0.025 | 10 | 10.2 | 1.600 | 18.750 | 2.941 |
| | | | 140 | | | | | | Total | 8.333 |

Results:

Excel spreadsheet attached with diarrhea scores and weights for animals irradiated with 14 or 14.5Gy. Because the data from each radiation dose were very similar, only the analysis of the animals irradiated with 14Gy is provided.

Weights: Mass specific growth rate was calculated by:

$$\frac{ln ( {M}_{f} ) - ln ( {M}_{i} )}{{T}_{f} - {T}_{i}} = MSGR$$

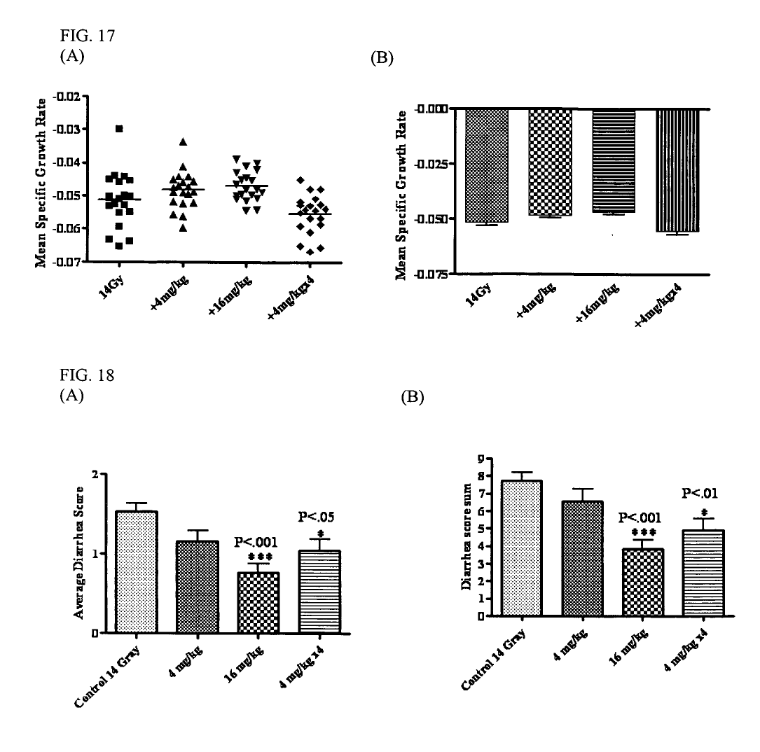

Significance was calculated using One-way ANOVA and Dunnett's Multiple Comparison Test. No significant differences were seen between the changes in weight during the study between the groups (FIGS. 17A and 17B).

Diarrhea score: Mice were scored for severity of diarrhea on a scale of 0-3 twice a day for three days beginning at 4 days after irradiation. Average diarrhea score over three days as well as the sum of the diarrhea score over three days was measured and graphed. Significance was obtained by one-way ANOVA and Tukey's Multiple Comparison Test. (FIGS. 18A and 18B)

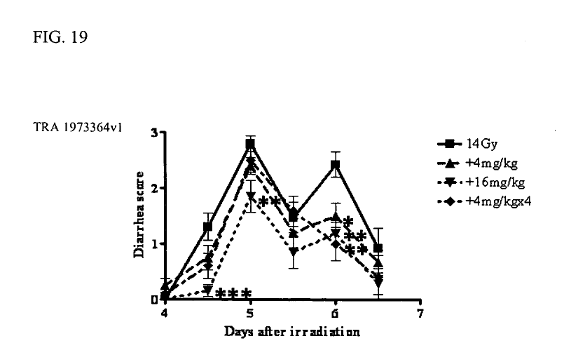

An analysis of for each day of observation was also made to determine differences at days of peak diarrhea. Significance was determined as described above (*−P<0.05, **−P<0.01, ***−P<0.001). (FIG. 19)

Conclusions:

Dosing animals with 16 mg/kg CG53135 at days −1, 0 and +1 respective to radiation resulted in a highly significant reduction in the incidence, length and severity of radiation-induced diarrhea. Dosing animals every 6 hours on day 1 with 4 mg/kg CG53135 also resulted in significant decrease in diarrhea incidence. The day of peak diarrhea was 5 days after radiation, at which point only the 16 mg/kg dose of CG53135 provided a significant decrease in diarrhea. There were no significant differences between the treatment groups in weight loss over the course of the study.

### 6.17 Example 17

### Manufacture of CG53135-05 and Pharmaceutical Formulations

Aiming for a construct that would be suitable for clinical development, untagged molecules were generated in a phage-free bacterial host. The codon-optimized, full-length, untagged molecule (CG53135-05) has the most favorable pharmacology profile and was used to prepare product for the safety studies and clinical trial.

### 6.17.1 Production Process and Pharmaceutical Formulations (Process 1)

CG53135-05 was expressed in Escherichia coli BLR (DE3) using a codon-optimized construct, purified to homogeneity, and characterized by standard protein chemistry techniques. The isolated CG53135-05 protein migrated as a single band (23 kilodalton) using standard SDS-PAGE techniques and stained with Coommassie blue. The CG53135-05 protein was electrophoretically transferred to a polyvinylidenefluoride membrane and the stained 23 kD band was excised from the membrane and analyzed by an automated Edman sequencer (Procise, Applied Biosystems, Foster City, Calif.); the N-terminal amino acid sequence of the first 10 amino acids was confirmed as identical to the predicted protein sequence.

Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100-110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

The finished drug product is a sterile, clear, colorless solution in single-use sterile vials for injection. CG53135-05 E. coli purified product was formulated at a final concentration of 8.2 mg/mL (Table 27).

TABLE 27
Composition of Drug Product

| | Grade | Final concentration | Amount per Liter |
|---|---|---|---|
| Component | | | |
| CG53135-05 E. coli | NA | 8.2 mg/ml | 8.2 g |
| purified product | | | |
| Formulation Buffer | | | |
| Sodium acetate | USP | 40 mM | 5.44 g |
| (trihydrate) | | | |
| L-arginine HCl | USP | 200 mM | 42.132 g |
| Glycerol | USP | 3% v/v | 30 mL |
| Acetic acid | USP | NA | QS to pH 5.3 |
| Water for injection | USP | NA | QS to 1 L |

The pharmacokinetics of optimally-formulated CG53135-05 E. coli purified product was assessed in rats following intravenous, subcutaneous, and intraperitoneal administration to compare exposure at active doses in animal models and predict exposure in humans. Intravenous administration of CG53135-05 E. coli purified product resulted in high plasma levels (maximum plasma level=19,680-47,252 ng/mL), which rapidly declined within the first 2 hours to 30-70 ng/mL; decreased exposure was observed following the third daily dose (maximum plasma level=5373-7453 ng/mL). Subcutaneous administration of CG53135-05 E. coli purified product resulted in slow absorption (maximum plasma level at 10 hours) and plasma levels of 40-80 ng/mL up to 48 hours after dosing; some accumulation in plasma was seen following the third daily dose. Intraperitoneal administration of CG53135-05 E. coli purified product resulted in slow absorption (maximum plasma level at 2-4 hours) and plasma levels of 40-70 ng/mL up to 10 hours after dosing; decreased exposure was seen following third daily dose. No significant gender differences were observed by any route of administration.

Safety of intravenous administration of CG53135-05 E. coli purified product (0.05, 5 or 50 mg/kg/day (Bradford) for 14 consecutive days) was assessed in a pivotal toxicology study in rats. There were no treatment-related findings in rats administered 0.05 mg/mL (Bradford) CG53135-05 E. coli purified product for 14 days. In rats administered 5 mg/kg (Bradford) CG53135 for 14 days, food consumption was reduced and body weight was decreased; while there were no treatment-related changes in organ weights, urinalysis, ophthalmology, or histopathology parameters in this dose group, there were treatment-related changes in hematology and clinical chemistry parameters in this treatment group. In rats administered 50 mg/kg (Bradford) CG53135-05 E. coli purified product for 12 days (estimated maximum plasma level of 20-30 fold higher than active dose), food consumption was reduced and body weight was markedly decreased; while there were no treatment-related changes in ophthalmology, there were significant treatment-related changes in organ weights, urinalysis, hematology, clinical chemistry, and histopathology in this treatment group.

Safety of intravenous administration of CG53135-05 E. coli purified product (0 or 10 mg/kg/day (Bradford) for 7 consecutive days) was further assessed in a safety pharmacology study in rhesus monkeys. There were no treatment-related clinical observations in animals administered 1 mg/kg (Bradford) CG53135-05 E. coli purified product for 7 days. In animals administered 10 mg/kg (Bradford) CG53135-05 E. coli purified product for 7 days, minor effects on body weight were noted and associated with qualitative observations of lower food consumption. There were no apparent treatment-related effects on hematology, clinical chemistry, ophthalmology, or electrophysiology in either dose group.

Stability of CG53135-05 Drug Substances

Stability studies on the CG53135-05 E. coli purified product produced during cGMP manufacturing were performed. The analytical methods used as stability indicating assays for purified drug substance are listed in Table 28.

TABLE 28
Stability Assays for Drug Substance

| Assay | Stability Criteria |
|---|---|
| SDS-PAGE | >98% pure by densitometry (reduced and |
| (Neuhoff stain) | nonreduced) |
| RP-HPLC | Peak at 5.5 ± 1.0 min relative retention time |
| SEC-HPLC | >90% mono-disperse peak |
| Total protein by | >0.2 mg/mL |
| Bradford method | |
| Bioassay (BrdU) | PI₂₀₀ > 0.5 ng/mL and <20 ng/mL |
| pH | 5.8 ± 0.4 |
| Visual appearance | Clear and colorless |

PI₂₀₀ = concentration of CG53135-05 that results in incorporation of BrdU at 2 times the background

The SDS-PAGE, RP-HPLC, and Bradford assays are indicative of protein degradation or gross aggregation. The SEC-HPLC assay detects aggregation of the protein or changes in oligomerization, and the bioassay detects loss of biological activity of the protein. The stability studies for the purified drug substance were conducted at −80 to 15° C. with samples tested at intervals of 3, 6, 9, 12, and 24 months.

In one experiment, stability studies of finished drug product were conducted by Cambrex at −80±15° C. and −20±5° C. with samples tested at intervals of 1, 3, 6, 9, 12, and 24 month data collected after 1 month indicate that finished drug product is stable for at least 1 month when stored at −80±15° C. or at −20±5° C. (Table 29).

TABLE 29
Stability Data for Drug Product after 1-month interval

| Assay | Stability Criteria | Initial | −80 ± 15° C. | −20 ± 5° C. |
|---|---|---|---|---|
| RP-HPLC | Major peak | Major peak | Major peak | Major peak |
| | retention time ± 0.2 min | retention time ± 0.2 min | retention time ± 0.2 min | retention time ± 0.2 min |
| | relative to | relative | relative | relative |
| | Reference | to Reference | to Reference | to Reference |
| | Standard | Standard | Standard | Standard |
| SDS-PAGE | Major band | Pass | Pass | Pass |
| | migrates at about | | | |
| | 23 kDa; | | | |
| | nonreduced minor | | | |
| | band below major | | | |
| | band | | | |
| SEC-HPLC | >90% mono-disperse | 100% | 100% | 100% |
| | peak | | | |
| Bradford | 10 ± 0.2 mg/mL | 8.2 | 8.6 | 8.3 |
| Bioassay | PI₂₀₀ > 0.5 ng/mL | 4.14 ng/mL | 2.98 ng/mL | 1/45 ng/mL |
| | and <20 ng/mL | | | |
| Sterility | Pass (ie., no | Pass | NT | NT |
| | growth) | | | |
| pH | 5.3 ± 0.3 | 5.4 | 5.5 | 5.4 |
| Visual | Clear and colorless | Pass | Pass | Pass |
| appearance | solution | | | |

Lot #02502001 was stored at −80±15° C. or at −20±5° C. at Cambrex and tested after month; Pl200=concentration of CG53135-05 that results in incorporation of BrdU at 2 times the background; Pass=results met stability criterion; NT=not tested

In another experiment, samples of finished drug product were stored at −80±15° C. or stressed at 5±3° C., 25±2° C., or 37±2° C. and tested at various intervals for 1 month. Stability data indicate that finished drug product showed no significant instability after 1 month of storage at −80±15OC or 5 ±3° C. When stressed at 25±2° C., finished drug product was stable for at least 48 hours; degradation was apparent after 1 week at this temperature. When stressed at 37±2° C., degradation of finished drug product was apparent within 4 hours.

### 6.17.2 Improved Pharmaceutical Formulations and Production Process of CG53135-05 (Process 2)

A new formulation was developed to meet the three requirements for a commercial product: (1) the minimal storage temperature should be 2-8° C. for ease of distribution; (2) product should be stable at the storage temperature for at least 18 months for a commercial distribution system; and (3) product should be manufactured by commercial scale equipment, and processes should be transferable to various commercial contract manufacturers.

The new formulation consists 10 mg/mL (UV) of the protein product produced by the process described in Section 6.2 (“Process 2 protein”) in 0.5 M arginine as sulfate salt, 0.05 M sodium phosphate monobasic, and 0.01% (w/v) polysorbate 80. The lyophilized product is projected to be stable for at least 18 months at 2-8° C. based on accelerated stability data. In contrast to the new formulation, the previous formulation as described in U.S. application Ser. No.10/435,087 is not possible to be lyophilized for the following reasons: firstly, the acidic component of the acetate buffer is acetic acid, which sublimes during lyophilization. After lyophilization, the loss of acetic acid is at 100% level with the basic component, sodium acetate, being the only buffering agent. This loss of acetic acid to lyophilization increases the pH to >7.5, which is far from the target pH of 5.3. Secondly, the glycerol has a collapse temperature of <−45° C., which renders this formulation not be able to be lyophilized commercially. Most of the commercial lyophilizers have a shelf temperature ranged from −45° C. to −50° C. with temperature variation of ±3° C.

Four unexpected properties of CG53135 were discovered and used to develop the new formulation: (1) high concentration of arginine, >0.4 M, increases the solubility to >30 mg/mL; (2) the use of sulfate salt of arginine increases the solubility by at least 2-6 fold; (3) the optimal concentration of sodium phosphate as a buffering salt is 50 mM, with a solubility of at least 1-2 fold increase in comparison with concentrations at 25, 75, and 100 mM; and (4) adding a surfactant during the diafiltration/ultrafiltration step minimizes the formation of aggregates. In development of the lyophilized formulation, each component of the new formulation was evaluated for solubility individually. CG53135-05 was precipitated using the precipitate buffer (50 mM NaPi, 5 mM EDTA, 1 M L-Arginine HCl, 2.5 M (NH₄)₂SO₄. The precipitate was washed with 25 mM sodium phosphate buffer at pH 6.5 to remove the residual arginine and ammonium sulfate. The washed precipitate was then re-dissolved in the following respective buffers listed in the tables. The following are examples of data.

TABLE 30
High concentration of arginine, >0.4 M, increases the solubility
to >30 mg/mL

| | Solubility of Process 2 protein in mg/mL | | | | |
|---|---|---|---|---|---|
| Concenctration of | | Batch | | | |
| Arginine (M) | Batch #1 | #2 | Batch #3 | Batch #4 | Batch #5 |
| 0.05 | 0.7 | 0.6 | 0.5 | ND | ND |
| 0.10 | 1.4 | 0.6 | 1.2 | ND | ND |
| 0.15 | 2.2 | 1.6 | 2.2 | ND | ND |
| 0.20 | 3.0 | 4.7 | 4.3 | ND | ND |
| 0.30 | ND | ND | ND | 5.8 | ND |
| 0.35 | ND | ND | ND | 10.1 | ND |
| 0.40 | ND | ND | ND | 9.8 | ND |
| 0.45 | ND | ND | ND | 32.3 | ND |
| 0.50 | ND | ND | ND | 23.8* | 37 |

*The solubility was lower as there was not sufficient protein in the experiment to be dissolved.

TABLE 31
The use of sulfate salt of arginine increases the solubility by at least
2-6 folds.

| Concentration of | Solubility of Process 2 protein in mg/mL | | | | |
|---|---|---|---|---|---|
| sodium phosphate | Batch | | | | |
| monobasic* | #A | Batch #B | Batch #C | Batch #D | Batch #E |
| 100 mM | 3.78 | 2.8 | 2.4 | 2.9 | 2.47 |
| 75 mM | 4.06 | 2.5 | 2.6 | 3.0 | 2.38 |
| 50 mM | 5.47 | 4.7 | 3.3 | 4.3 | 4.81 |
| 25 mM | 4.01 | 2.4 | 2.6 | 2.4 | 3.59 |

All formulation contains 0.2 M arginine.

An optimal concentration of the sodium phosphate as a buffering salt was observed (Table 32). The optimal concentration of sodium phosphate is 50 mM with a solubility of at least 1-2 fold increase in comparison with concentrations at 25, 75, and 100 Mm.

TABLE 32
The optimal concentration of sodium phosphate as a buffering salt
is 50 mM

| | Solubility Increament of Process 2 | |
|---|---|---|
| | protein in using Arginine Sulfate vs | |
| | Arginine Phosphate in mg/mL | |
| Formulation | Batch #K | Batch # J |
| 50 mM sodium phosphate | 4.4 | 2.3 |
| monobasic and 0.15M | | |
| Arginine at pH 7 | | |
| 50 mM sodium phosphate | 6.5 | 5.2 |
| monobasic and 0.15M | | |
| Arginine at pH 7 | | |

Table 33 shows a need to add a surfactant during the diafiltration/ultrafiltration step to minimize the formation of aggregates. The experiment was conducted by performing the ultrafiltration/diafiltration at 2.5 mg/mL CG53135-05 in 0.2M arginine and 0.05 M sodium phosphate buffer at pH 7.0. After exchanging with 7 volumes of the final buffer (0.5M arginine and 0.05 M sodium phosphate buffer at pH 7.0), the diafiltrate is concentrated to ˜20 mg/mL. The diafiltrate is then diluted with the final buffer to ˜12.5 mg/mL and lyophilized. Polysorbate 80 is added either before or after the diafiltration to a final concentration of 0.01 %.

TABLE 33
Adding a surfactant during the diafiltration/ultrafiltration step
minimizes the formation of aggregates.

| Polysorbate added | Process 2 protein | |
|---|---|---|
| during | Concentration | |
| ultrafiltration/diafiltration | (mg/mL) | Turbidity (NTU) |
| Yes | 12.5 | 20.9 |
| No | 13.0 | 4.6 |

All formulation contains 0.5 M arginine, 0.05 M sodium phosphate monobasic, and 0.01% polysorbate 80.

The new formulation has the following advantages: (1) a lyophilized product with a storage temperature of 2-8° C.; (2) a lyophilized product with a projected shelf-life of at least 18 months when stored at 2-8° C. achieve the solubility of >30 mg/mL; and (3) The lyophilized product has a collapse temperature of −30° C. which can be easily lyophilized by the commercial equipment. The interactions between arginine, sulfate, phosphate, and surfactant and CG53135 were unexpected.

The improved process steps for the manufacturing of drug substance and drug product are described in Table 34, and each step is explained below.

TABLE 34
Manufacturing Process
Ampoule from WCB
↓
Seed Flask and Seed Fermenter 25 L - Innoc
↓
Fermentation at 1500L scale
↓
Homogenization + 0.033% PEI or a charged
heterogenous polymer
↓
Purification by SP Streamline
↓
Purification by PPG 650M
↓
Cuno Filtration
↓
Purification by Phenyl Sepharose HP
↓
Concentration/Diafiltration addition of 0.01%
polysorbate 80 or Polysorbate 20
↓
Bottling - Drug Substance
↓
QC Testing and Release
↓
Sterile Vial Fill & Lyophilization
↓
Drug Product
↓
QC Testing and Release

Cell Bank: a Manufacturing Master Cell Bank (MMCB) in animal component free complex medium was used in an earlier Process. A second Manufacturing Master Cell Bank (MMCB) in animal component free chemically-defined medium was derived from the first MMCB and a Manufacturing Working Cell Bank (MWCB) was made from the second MMCB. This MWCB was used in the manufacturing process as described in Table 34.

Inoculum Preparation: the initial cell expansion occurs in shake flasks. Seed preparation is done by thawing and pooling 2-3 vials of the MWCB in chemically defined medium and inoculating 3-4 shake flasks each containing 500 mL of chemically-defined seed medium.

Seed and Final Fermentation: the shake flasks with cells in exponential growth phase (2.5-4.5 OD600 units) are used to inoculate a single 25 L (i.e., working volume) seed fermenter containing the seed medium. The cells upon reaching exponential growth phase (3.0-5.0 OD600 units) in the 25 L seed fermenter are transferred to a 1500 L production fermenter with 780-820 L of chemically defined batch medium. During fermentation, the temperature is controlled at 37±2° C., pH at 7.1±0.1, agitation at 150-250 rpm and sparging with 0.5-1.5 (vvm) of air or oxygen-enriched air to control dissolved oxygen at 25% or above. Antifoam agent (Fermax adjuvant 27) is used as needed to control foaming in the fermenter. When the OD (at 600 nm) of culture reaches 25-35 units, additional chemically defined medium is fed at 0.7 g/kg broth/min initially and then with feed rate adjustment as needed. The induction for expression of CG53135-05 protein is started when OD at 600 nm reaches 135-165 units. After 4 hours post-induction the fermentation is completed. The final fermentation broth volume is approximately 1500 L. The culture is then chilled to 10-15° C.

Homogenization: the chilled culture is diluted with cell lysis buffer at the ratio of one part of fermentation broth to two parts of cell lysis buffer (50 mM sodium phosphate, 60 mM EDTA, 7.5 mM DTT, 4.5 M urea, pH 7.2. Polyethyleneimine (PEI), a flocculating agent is added to the diluted fermentation broth to a final PEI concentration at 0.033% (WN). The cells are lysed at 10-15° C. with 3 passages through a high-pressure homogenizer at 750-850 bar.

Capture and Recovery: the chilled cell lysate is directly loaded in the upflow direction onto a pre-equilibrated Streamline SP expanded bed cation exchange column. During the loading, the bed expansion factor is maintained between 2.5-3.0 times the packed bed column volume. After loading, the column is flushed with additional Streamline SP equilibration buffer (100 mM sodium phosphate, 40 mM EDTA, 10 mM sodium sulfate, 3 M urea, pH 7.0) in the upflow direction. The column is then washed further with SP Streamline wash buffer (100 mM sodium phosphate, 5 mM EDTA, 25 mM sodium sulfate, 2.22 M dextrose, pH 7.0) in the downflow direction. The protein is eluted from the column with Streamline SP elution buffer (100 mM sodium phosphate, 5 mM EDTA, 200 mM sodium sulfate, 1 M L-arginine, pH 7.0) in the downflow direction.

PPG 650M Chromatography: the SP Streamline eluate is loaded on to a pre-equilibrated PPG 650 M, hydrophobic interaction chromatography column. The column is equilibrated and washed with 100 mM sodium phosphate, 200 mM sodium sulfate, 5 mM EDTA, 1 M Arginine pH 7.0. The column is further washed with 100 mM sodium phosphate, 5 mM EDTA, 0.9 M Arginine, pH 7.0. The product is eluted with 100 mM sodium phosphate, 5 mM EDTA, 0.2 M Arginine, pH 7.0.

CUNO Filtration: the PPG eluate is passed through an endotoxin binding CUNO 30ZA depth filter. The filter is flushed first with water for injection (WFI) and then with 100 mM sodium phosphate, 5 mM EDTA, 0.2 M Arginine, pH 7.0 (PPG eluate buffer). After flushing, the PPG eluate is passed through the filter. Air pressure is used to push the final liquid through the filter and its housing.

Phenyl Sepharose Chromatography: the CUNO filtrate is then loaded on to a pre-equilibrated Phenyl Sepharose hydrophobic interaction chromatography column. The column is equilibrated and washed with 100 mM sodium phosphate, 50 mM ammonium sulfate, 800 mM sodium chloride, 0.5 M Arginine, pH 7.0. The product is eluted with 50 mM sodium phosphate, 0.5 M Arginine, pH 7.0.

Concentration and Diafiltration: a 1% Polysorbate 80 is added to the Phenyl Sepharose eluate so that the final concentration in the drug substance is 0.01% (w/v). The eluate is then concentrated in an ultrafiltration system to about 2-3 g/L. The retentate is then diafiltered with 7 diafiltration volumes of 50 mM sodium phosphate, 0.5 M Arginine, pH 7.0 (Phenyl Sepharose elution buffer). After diafiltration the retentate is concentrated between 12-15 g/L. The retentate is filtered through a 0.22 μm filter and subsequently diluted to 10 g/L.

Bulk Bottling: the retentate from the concentration and diafiltration step is filtered through a 0.22 μm pore size filter into 2 L single use Teflon bottles. The bottles are frozen at −70° C.

Drug Product/Vial: the bottles of frozen Drug Substance are thawed at ambient temperature. After the Drug Substance is completely thawed, it is pooled in a sterile container, filtered, filled into vials, partially stoppered, and lyophilized. After completion of the freeze-drying process, the vials are stoppered and capped. The lyophilized Drug Product is stored at 2-8° C.

The CG53135-05 reference standard was prepared at Diosynth RTP Inc, using a 140 L scale manufacturing process that was representative of the bulk drug substance manufacturing process (as described in the General Method of Manufacture). The reference standard was stored as 1 mL aliquots in 2 mL cryovials at −80° C.±15° C.

Purity of the final product was analyzed by SDS-PAGE, RP-HPLC, size exclusion-HPLC, and Western blot. Potency of the drug was measured by growth of NIH 3T3 cells in response to CG53135-05 E. coli purified product. All data indicated that the final product is suitable for clinical uses.

### 7. Equivalents

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

1. A method of preventing or treating alimentary mucositis comprising administering to a subject in need thereof a prophylactically or therapeutically effective amount of an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

2. A method of preventing or treating alimentary mucositis comprising administering to a subject in need thereof a prophylactically or therapeutically effective amount of a protein isolated from a cultured host cell containing an isolated nucleic acid molecule selected from the group consisting of:
(a) a nucleic acid molecule comprising a nucleotide sequence selected from the group consisting of SEQ ID NOs: 1, 3, 5, 6, 8, 9, 21, 23, 25, 27, 29, 31, 33, 35, 37, 39 and 41;
(b) a nucleic acid molecule encoding a protein comprising an amino acid sequence selected from the group consisting of SEQ ID NOs: 2, 4, 7, 10, 22, 24, 26, 28, 30, 32, 34, 36, 38, or 40; and
(c) a nucleic acid molecule hybridizes under stringent conditions to a nucleotide sequence of SEQ ID NO: 1, 3, 5, 6, 8, 9, 21, 23, 25, 27, 29, 31, 33, 35, 37, 39 or 41, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA.

3. The method of claim 2, wherein said host cell is a eukaryotic cell.

4. The method of claim 2, wherein said host cell is a prokaryotic cell.

5. The method of claim 4, wherein said prokaryotic cell is E. coli.

6. The method of claim 2, wherein said protein isolated from a cultured host cell has a purity of at least 98%.

7. The method of claim 2, wherein said protein isolated from a cultured host cell has a purity of at least 99%.

8. A method of preventing or treating alimentary mucositis comprising administering to a subject in need thereof a prophylactically or therapeutically effective amount of a composition comprising a pharmaceutically acceptable carrier, and an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

9. The method of claim 8, wherein said composition comprising 0.04M sodium acetate, 3% Glycerol (volume/volume), 0.2M Arginine-HCl at pH 5.3, and 3 mg/ml of said isolated protein.

10. The method of claim 8, wherein said composition comprising 0.1-1 M arginine or a salt thereof, 0.01-0.1 M sodium phosphate monobasic (NaH₂PO₄·H₂O), 0.01% -0.1% weight/Volume (“w/v”) polysorbate 80 or polysorbate 20, and 2-50 mg/ml of said isolated protein.

11. The method of claim 10, wherein said arginine or a salt thereof is selected from the group consisting of arginine, arginine sulfate, arginine phosphate, and arginine hydrochloride.

12. The method of claim 10, wherein said arginine or a salt thereof is of 0.5 M.

13. The method of claim 10, wherein said sodium phosphate monobasic is 0.05 M.

14. The method of claim 10, wherein said polysorbate 80 or polysorbate 20 is 0.01% (w/v).

15. The method of claim 10, wherein said isolated protein is at a concentration of 5-30 mg/ml.

16. The method of claim 10, wherein said isolated protein is at a concentration of 10 mg/ml.

17. The method of claim 10, wherein said isolated protein comprises an amino acid sequence of SEQ ID NO:24.

18. The method of claim 10, wherein said isolated protein comprises an amino acid sequence of SEQ ID NO:2.

19. The method of claim 10, wherein said isolated protein comprises two or more proteins.

20. The method of claim 19, wherein said composition comprises a first protein comprising an amino acid sequence of SEQ ID NO:24, and a second protein comprising an amino acid sequence of SEQ ID NO:2.

21. The method of claim 20, wherein said composition further comprises an isolated protein comprising an amino acid sequence selected from the group consisting of SEQ ID NOs: 26, 28, 30 and 32.

22. The method of claim 20, wherein said composition further comprises a third protein comprising an amino acid sequence of SEQ ID NO:28, a fourth protein comprising an amino acid sequence of SEQ ID NO:30, and a fifth protein comprising an amino acid sequence of SEQ ID NO:32.

23. The method of claim 10, wherein said composition is lyophilized.

24. The method of claim 10, wherein said isolated protein has at least 98% purity.

25. The method of claim 1, 2 or 8, wherein said alimentary mucositis is oral mucositis.

26. The method of claim 1, 2 or 8, wherein said alimentary mucositis is enteritis.

27. The method of claim 1, 2 or 8, wherein said alimentary mucositis is esophagitis, stomatitis, or proctitis.

28. The method of claim 1, 2 or 8, wherein said alimentary mucositis is caused by a chemical insult, a biological insult, radiation, or a combination thereof.

29. The method of claim 1, 2 or 8, wherein said subject is a mammal.

30. The method of claim 29, wherein said mammal is a human.

31. The method of claim 1, 2 or 8, wherein said effective amount is between 0.001-3 mg/kg.

32. The method of claim 1, 2 or 8, wherein said effective amount is between 0.01,-1 mg/kg.

33. The method of claim 1, 2 or 8, wherein said effective amount is between 0.01-0.5 mg/kg.

34. The method of claim 1, 2 or 8, wherein said effective amount is about 0.03 mg/kg, about 0.1 mg/kg, about 0.2 mg/kg, about 0.5 mg/kg, about 1 mg/kg, about 2 mg/kg, or about 3 mg/kg.

35. The method of claim 1, 2 or 8, wherein said administering is a single dose administered at a dosage of 0.001-1 mg/kg, 0.01-0.5 mg/kg, 0.01-0.2 mg/kg, 0.03 mg/kg, 0.1 mg/kg, or 0.2 mg

36. The method of claim 1, 2 or 8, wherein said administering is a multiple dosing administered with each unit dosage of 0.001-0.5 mg/kg, 0.01-0.2 mg/kg, 0.03 mg/kg, 0.1 mg/kg, or 0.2 mg/kg.

37. The method of claim 1, 2 or 8, wherein said administering is parenteral administration.

38. The method of claim 37, wherein said parenteral administration is intravenous administration or subcutaneous administration.

39. The method of claim 1, 2 or 8, wherein said administering is rectal administration, transdermal administration, or transmucosal administration.

40. A method of producing an isolated protein comprising the steps of:
(1) fermenting an E. coli cell containing a vector comprising SEQ ID NO:8;
(2) suspending the cultured cells in a lysis buffer comprising 3 M urea;
(3) lysing the cultured cells;
(4) loading the clarified lysate onto a SP-sepharose Fast Flow column equilibrated with a buffer comprising 3 M urea, 100 mM sodium phosphate, 20 mM sodium chloride, 5 mM EDTA;
(5) loading the column with a buffer comprising 100 mM sodium citrate, 1 M arginine, 5 mM EDTA to elute a protein;
(6) filtering the resultant eluate;
(7) precipitate the filtered eluate with ammonium sulfate;
(8) solubilizing the precipitated eluate in a buffer comprising 100 mM sodium citrate, 500 mM argine, 750 mM NaCl, 5 mM EDTA;
(9) loading the solubilized eluate onto a Phenyl-seharose HP column;
(10) washing the column with a linear gradient with a buffer comprising 100 mM sodium citrate, 500 mM arginine, 5 mM EDTA to elute a protein.

41. An isolated protein obtained by the method of claim 40.

42. An aqueous formulation comprising a formulation buffer and an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity;
(c) an isolated protein of claim. 41; and
(d) a fragment of the protein of (a), (b) or (c), which fragment retains cell proliferation stimulatory activity.

43. The formulation of claim 42, wherein the formulation buffer comprises 0.02-0.2 M acetate, 0.5-5% glycerol (volume/volume), and 0.2-0.5 M arginine-HCl.

44. The formulation of claim 42, wherein the formulation buffer comprises 0.04 M sodium acetate, 0.2 M arginine-HCl, 3% glycerol (volume/volume).

45. The formulation of claim 42, wherein pH of the formulation is about 4.9 to about 6.2.

46. The formulation of claim 42, wherein pH of the formulation is about 5.3.

47. The formulation of claim 42, wherein said formulation is stable for at least one month at a temperature of 8° C. or lower.

48. The formulation of claim 42, wherein said formulation comprises 0.05 mg/ml to 10 mg/ml of said isolated protein.

49. The formulation of claim 42, wherein said formulation comprises 0.8 mg/ml of said isolated protein.


SEQUENCE LISTING (41 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact